(19) United States
(12) Patent Application Publication
(10) Pub. No.: US 2026/0055455 A1
(43) Pub. Date: Feb. 26, 2026

(54) METHODS AND COMPOSITIONS FOR NUCLEIC ACID SEQUENCING

(71) Applicant: 10x Genomics, Inc., Pleasanton, CA (US)

(72) Inventor: Justin COSTA, Union City, CA (US)

(21) Appl. No.: 19/251,423

(22) Filed: Jun. 26, 2025

Related U.S. Application Data
(60) Provisional application No. 63/665,774, filed on Jun. 28, 2024.

(51) Int. Cl. C12Q 1/6874; C07K 14/435; C12Q 1/48; C12Q 1/6834; C12Q 1/686; C12Q 1/6876

(52) U.S. Cl. CPC C12Q 1/6874; C07K 14/43563; C12Q 1/48; C12Q 1/6834; C12Q 1/686; C12Q 1/6876; C12Y 207/07; C07K 2319/00

(57) ABSTRACT

The present disclosure relates in some aspects to methods, systems, and kits for sequencing a template nucleic acid molecule using one or more polymerase-luciferase fusion proteins. In some embodiments, different types of polymerase-luciferase fusion proteins and are sequentially contacted with a template nucleic acid in the presence of different types of nucleotides under conditions that stabilize a ternary complex between a given polymerase-luciferase fusion protein, the template nucleic acid molecule, and a nucleotide when the nucleotide is complementary to the template nucleic acid. By imaging the sample to detect luminescence, the base of the template nucleic acid can be identified.

### CROSS-REFERENCE TO RELATED APPLICATIONS

This application claims benefit of and priority to U.S. Provisional Application No. 63/665,774, filed on Jun. 28, 2024, entitled “Methods and Compositions for Nucleic Acid Sequencing,” which is hereby incorporated by reference in its entirety.

### FIELD

The present disclosure relates in some aspects to methods for sequencing nucleic acid molecules, including methods for in situ sequencing and analysis of target nucleic acids in a biological sample.

### BACKGROUND

Nucleic acid sequencing is a versatile tool that helps scientists advance the understanding of biology and has wide-ranging applications in various fields, such as medical diagnostics, biotechnology, forensic biology, and virology. Currently, there are several sequencing methods available, including Maxam-Gilbert sequencing, Sanger (chain-termination) sequencing, and next-generation sequencing (NGS) techniques. Despite advances in nucleic acid sequencing, many challenges remain unaddressed.

### SUMMARY

NGS sequencing-by-synthesis (SBS) is based on incorporation of a fluorescent, reversibly terminated nucleotide into an extended priming strand, where the incorporated nucleotide is complementary to a nucleotide in the template nucleic acid molecule that is being probed. In such methods, each nucleotide is labelled with a single fluorophore which can limit sensitivity and signal intensity. However, in conventional SBS approaches, any incompleteness of cleavage-either at the level of the fluorophore or the reversible terminator group, will lead to phasing of the optical barcode signatures detected in the sequencing reaction. Additional challenges such as autofluorescence also exist for nucleic acid sequencing in cell or tissue samples. The present application addresses these and other needs. In some aspects, the use of polymerases labeled with luciferase proteins capable of emitting distinguishable luminescent signals in the presence of substrate avoids problems with autofluorescence in cell or tissue samples, because no excitation light is required to detect the luminescent signals (unlike conventional fluorescent signals). In some aspects, the nucleotides themselves are not fluorescently labeled, avoiding problems related to incomplete cleavage of labels from incorporated nucleotides. In some embodiments, nucleotides that do not comprise reversible terminators are used, avoiding problems related to incomplete cleavage of reversible terminators.

In some embodiments, provided herein is a method for sequencing a template nucleic acid molecule comprising: (a-i) contacting a sample comprising a priming strand bound to the template nucleic acid molecule with a first type of polymerase-luciferase fusion protein and a first type of nucleotide under conditions that stabilize a ternary complex between the first type of polymerase-luciferase fusion protein, the template nucleic acid molecule, and the first type of nucleotide when the first type of nucleotide is complementary to the template nucleic acid at a base adjacent to the bound priming strand; (a-ii) contacting the sample with a second type of polymerase-luciferase fusion protein and a second type of nucleotide under conditions that stabilize a ternary complex between the second type of polymerase-luciferase fusion protein, the template nucleic acid molecule, and the second type of nucleotide when the second type of nucleotide is complementary to the template nucleic acid at the base adjacent to the bound priming strand; and (b) imaging the sample to detect luminescence, wherein: the first type of polymerase-luciferase fusion comprises a first type of luciferase that emits a first type of luminescent signal indicating formation of a ternary complex comprising the first type of nucleotide, and the second type of polymerase-luciferase fusion comprises a second type of luciferase that emits a second type of luminescent signal indicating formation of a ternary complex comprising the second type of nucleotide; thereby identifying the base of the template nucleic acid adjacent to the bound priming strand from the detected luminescence.

In some embodiments, the method further comprises: (a-iii) contacting the sample with a third type of polymerase-luciferase fusion protein and a third type of nucleotide under conditions that stabilize a ternary complex between the third type of polymerase-luciferase fusion protein, the template nucleic acid molecule, and the third type of nucleotide when the third type of nucleotide is complementary to the template nucleic acid at the base adjacent to the bound priming strand wherein the third type of polymerase-luciferase fusion comprises a third type of luciferase that emits a third type of luminescent signal indicating formation of a ternary complex comprising the third type of nucleotide.

In some embodiments, the method further comprises: (a-iv) contacting the sample with a fourth type of nucleotide and a polymerase under conditions that stabilize a ternary complex between the polymerase, the template nucleic acid molecule, and the fourth type of nucleotide when the fourth type of nucleotide is complementary to the template nucleic acid at the base adjacent to the bound priming strand.

In some embodiments, the method further comprises: (a-iv) contacting the sample with a fourth type of polymerase-luciferase fusion protein and a fourth type of nucleotide under conditions that stabilize a ternary complex between the fourth type of polymerase-luciferase fusion protein, the template nucleic acid molecule, and the fourth type of nucleotide when the fourth type of nucleotide is complementary to the template nucleic acid at the base adjacent to the bound priming strand; wherein the fourth type of polymerase-luciferase fusion comprises a fourth type of luciferase that emits a fourth type of luminescent signal indicating formation of a ternary complex comprising the fourth type of nucleotide.

In some embodiments, the first and second types of luciferase are different types of luciferase independently selected from the group consisting of a yellow-emitting luciferase, a green-emitting luciferase, a blue-emitting luciferase, a red-emitting luciferase, and a far-red-emitting luciferase. In some embodiments, the first, second, and third types of luciferase are different types of luciferase independently selected from the group consisting of a yellow-emitting luciferase, a green-emitting luciferase, a blue-emitting luciferase, a red-emitting luciferase, and a far-red-emitting luciferase. In some embodiments, the first, second, third, and fourth types of luciferase are different types of luciferase independently selected from the group consisting of a yellow-emitting luciferase, a green-emitting luciferase, a blue-emitting luciferase, a red-emitting luciferase, and a far-red-emitting luciferase.

In some embodiments, the ternary complex-stabilizing conditions are maintained throughout steps (a-i), (a-ii), and step (b). In some embodiments, the ternary complex-stabilizing conditions are maintained throughout steps (a-i), (a-ii), (a-iii), and step (b). In some embodiments, the ternary complex-stabilizing conditions are maintained throughout steps (a-i) through (a-iv) and step (b).

In some embodiments, the ternary complex-stabilizing conditions comprise presence of a non-catalytic metal ion. In some embodiments, the ternary complex-stabilizing conditions comprise absence of a catalytic metal ion cofactor of the polymerase. In some embodiments, the catalytic metal ion is magnesium or manganese. In some embodiments, the non-catalytic metal ion is calcium, strontium, tin, or nickel.

In some embodiments, the method comprises washing the sample to remove unbound polymerase-luciferase fusion proteins and unbound nucleotides prior to performing the detecting step. In some embodiments, the washing is performed with a wash buffer comprising a non-catalytic metal ion, optionally wherein the non-catalytic metal ion is calcium, strontium, tin, or nickel. In some embodiments, the method comprises washing the sample between each of the contacting steps to remove unbound polymerase-luciferase fusion proteins and unbound nucleotides. In some embodiments, the washing is performed with a wash buffer comprising a non-catalytic metal ion, optionally wherein the non-catalytic metal ion is calcium, strontium, tin, or nickel. In some embodiments, the washing is performed with a wash buffer that does not comprise a catalytic metal cation of the polymerase.

In some embodiments, after imaging the sample to detect luminescence, the method comprises extending the priming strand by incorporating a reversibly terminated nucleotide, and repeating steps (a-i), (a-ii), and (b) for at least one additional cycle to identify at least one additional base of the template nucleic acid strand. In some embodiments, the at least one additional cycle comprises at least 2, 5, 10, 20, 30, 40, or 50 additional cycles.

In some embodiments, after imaging the sample to detect luminescence, the method comprises extending the priming strand by incorporating a reversibly terminated nucleotide, and repeating steps (a-i), (a-ii), (a-iii), (a-iv), and (b) for at least one additional cycle to identify at least one additional base of the template nucleic acid strand. In some embodiments, the at least one additional cycle comprises at least 2, 5, 10, 20, 30, 40, or 50 additional cycles.

In some embodiments, the priming strand is reversibly terminated, and the method comprises deprotecting the reversible terminator before extending the priming strand.

In some embodiments, the first type of nucleotide, second type of nucleotide, third type of nucleotide, and fourth type of nucleotide comprise different nucleobases selected from the group consisting of A, T, C, and G. In some embodiments, the first type of nucleotide, second type of nucleotide, third type of nucleotide, and fourth type of nucleotide comprise different nucleobases selected from the group consisting of A, T or U, C, and G. In some embodiments, the first type of nucleotide and second type of nucleotide are not reversibly terminated nucleotides. In some embodiments, the first type of nucleotide, second type of nucleotide, and third type of nucleotide are not reversibly terminated nucleotides. In some embodiments, the first type of nucleotide, second type of nucleotide, third type of nucleotide, and fourth type are not reversibly terminated nucleotides. In some embodiments, the first type of nucleotide, second type of nucleotide, third type of nucleotide, and fourth type are reversibly terminated nucleotides.

In some embodiments, the template nucleic acid molecule comprises a DNA molecule. In some embodiments, the template nucleic acid molecule comprises an RNA molecule. In some embodiments, the template nucleic acid molecule comprises a target analyte nucleic acid molecule.

In some embodiments, the template nucleic acid molecule comprises a sequence of interest associated with a target analyte. In some embodiments, the method comprises hybridizing a circularizable probe to the target analyte or to a labeling agent bound to the target analyte and ligating the circularizable probe to form a circularized probe, wherein the method further comprises performing rolling circle amplification of the circularized probe to generate the template nucleic acid molecule. In some embodiments, the circularizable probe is a padlock probe sequence. In some embodiments, the target analyte nucleic acid molecule comprises an mRNA molecule.

In some embodiments, the template nucleic acid molecule to be sequenced is attached to a solid support. In some embodiments, the solid support comprises a sequencing flow cell.

In some embodiments, the template nucleic acid molecule is sequenced in situ in a cell sample or tissue sample. In some embodiments, the cell sample comprises a layer of cells deposited on a surface.

In some embodiments, provided herein is a kit for sequencing a template nucleic acid molecule comprising: a first type of polymerase-luciferase fusion protein and a first type of nucleotide, a second type of polymerase-luciferase fusion protein and a second type of nucleotide, wherein the first type of polymerase-luciferase fusion comprises a first type of luciferase that emits a first type of luminescent signal and the second type of polymerase-luciferase fusion comprises a second type of luciferase that emits a second type of luminescent signal.

In some embodiments, the kit further comprises a third type of polymerase-luciferase fusion protein and a third type of nucleotide, wherein the third type of polymerase-luciferase fusion comprises a third type of luciferase that emits a third type of luminescent signal indicating formation of a ternary complex comprising the third type of nucleotide. In some embodiments, the kit further comprises a fourth type of nucleotide and a polymerase. In some embodiments, the kit further comprises a fourth type of polymerase-luciferase fusion protein and a fourth type of nucleotide, wherein the fourth type of polymerase-luciferase fusion comprises a fourth type of luciferase that emits a fourth type of luminescent signal indicating formation of a ternary complex comprising the fourth type of nucleotide.

In some embodiments, provided herein is a system comprising: one or more processors; and a memory communicatively coupled to the one or more processors and configured to store instructions that, when executed by the one or more processors, cause the system to perform the method of any of the preceding embodiments.

### BRIEF DESCRIPTION OF THE DRAWINGS

The drawings illustrate certain features and advantages of this disclosure. These embodiments are not intended to limit the scope of the appended claims in any manner.

FIG. 1 provides a schematic illustration of an example of a method comprising sequentially flowing different types of polymerase-luciferase fusion proteins into a biological sample together with corresponding different types of nucleotides under conditions that stabilize a ternary complex between a polymerase-luciferase fusion protein, template nucleic acid molecule, and nucleotide when the nucleotide is complementary to the template nucleic acid molecule at a position adjacent to a bound priming strand. After the different polymerases-luciferase fusion proteins bind, the sample is imaged to detect luminescent signals corresponding to different nucleotides and corresponding polymerase-luciferase fusion proteins bound to different template nucleic acids in the sample.

FIG. 2 depicts a system for performing a sequencing assay, in accordance with some implementations of the methods described herein.

FIG. 3 depicts a computer system or computer network, in accordance with some instances of the systems described herein.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

The present application leverages the wide availability of bioluminescent biomolecules capable of emitting luminescent signals at different optically distinguishable wavelengths for nucleic acid sequencing, including sequencing in situ in cell or tissue samples. In some embodiments, four different polymerase-luciferase fusion constructs (one for each color) are used. The polymerase-luciferase fusion constructs comprise a polymerase fused to a luciferase. In some embodiments, each of the four different types of luciferase is active with a different substrate. In some embodiments, each luciferase produces a different color. For 2-color or 3-color sequencing chemistries, in some embodiments an unlabeled polymerase is used for a “dark” signal. For 2 color sequencing chemistry, in some embodiments a composition comprising a mixture of two different polymerase-luciferase fusion proteins is contacted with the biological sample together with a third type of nucleotide, wherein detecting a luminescent signal from the first type of luciferase and the second type of luciferase colocalized in the biological sample indicates binding of the third type of nucleotide to a plurality of copies of a template nucleic acid sequence at that location.

In some embodiments, each of the different polymerase-luciferase fusion constructs (or a composition comprising a mixture of polymerase-luciferase fusion constructs or a “dark” polymerase) are introduced, one at a time, in the presence of just one known type of nucleotide (e.g., the red luciferase construct is introduced with dCTP). In some embodiments, the polymerase-luciferase fusion constructs (or a composition comprising a mixture of polymerase-luciferase fusion constructs or a “dark” polymerase) and corresponding nucleotides are contacted with the biological sample under conditions that promote formation of a ternary complex between the polymerase or polymerase-luciferase fusion protein, the template nucleic acid molecule, and the nucleotide when the nucleotide is complementary to the template nucleic acid at the base adjacent to the bound priming strand. In some embodiments, the buffer comprises a non-catalytic ion such as calcium instead of magnesium leading to a stalled ternary complex between the sequencing primer on the template nucleic acid (e.g., an RCP), the incoming nucleotide and the polymerase domain of the fusion construct. In some embodiments, after all four fusion/dNTP mixtures have been added, a mixture of the luminescent substrates is added, and luminescent signals is detected from the bound polymerase-luciferase fusion constructs. Each polymerase-luciferase fusion protein luminesces in the color corresponding to the matching nucleotide.

In some embodiments, after imaging, magnesium is bathed over the sample (all free nucleotides have been removed) and the trapped nucleotides in the ternary complex are then incorporated and the cycle can begin again. In some aspects, the use of polymerases labeled with luciferase proteins capable of emitting distinguishable luminescent signals in the presence of substrate avoids problems with autofluorescence in cell or tissue samples, because no excitation light is required to detect the luminescent signals (unlike conventional fluorescent signals). In some aspects, the nucleotides themselves are not fluorescently labeled, avoiding problems related to incomplete cleavage of labels from incorporated nucleotides. In some embodiments, nucleotides that do not comprise reversible terminators are used, avoiding problems related to incomplete cleavage of reversible terminators.

In some aspects, the use of polymerase-luciferase fusion proteins for sequencing according to the present disclosure provides numerous advantages. In some embodiments, the method does not comprise exposing the biological sample to a fluorescent excitation wavelength for any or for at least a subset of the cycles of base-by-base sequencing. Without being bound by theory, in some embodiments, performing nucleic acid sequencing without fluorescent excitation reduces or eliminates singlet oxygen induced crosslinking. In some embodiments, the buffers used for nucleic acid sequencing according to the present disclosure do not comprise oxygen scavengers. In some embodiments, the method avoids problems with photobleaching that may occur in conventional nucleic acid sequencing using fluorescent labels.

Additional aspects of the methods, compositions, kits, and systems disclosed herein are described in the sections below.

### II. Compositions

In some embodiments, provided herein is a composition for sequencing a template nucleic acid molecule comprising a polymerase-luciferase fusion protein as described herein. In some embodiments, provided herein is a composition comprising a single type of polymerase-luciferase fusion protein and a single type of nucleotide, wherein the nucleotide is selected such that the type of nucleotide corresponds to a color of luminescence emitted by the type of polymerase-luciferase fusion protein in the composition (in the presence of substrate). In some embodiments, the composition comprises a buffer comprising a non-catalytic metal ion. In some embodiments, the non-catalytic metal ion is calcium, strontium, tin, or nickel. In some embodiments, the buffer does not comprise a catalytic metal ion cofactor of the polymerase. In some embodiments, the buffer does not comprise magnesium or manganese.

In some embodiments (e.g., for use in a method of nucleic acid sequencing using two or three different luminescent colors to identify four different types of nucleotides), provided herein is a composition comprising two different types of polymerase-luciferase fusion protein and a single type of nucleotide, wherein the nucleotide is selected such that the type of nucleotide corresponds to a detection of the two colors of luminescence emitted by the two types of polymerase-luciferase fusion protein in the composition (in the presence of substrate(s)). In some embodiments, the composition comprises a buffer comprising a non-catalytic metal ion. In some embodiments, the non-catalytic metal ion is calcium, strontium, tin, or nickel. In some embodiments, the buffer does not comprise a catalytic metal ion cofactor of the polymerase. In some embodiments, the buffer does not comprise magnesium or manganese.

In some embodiments (e.g., for use in a method of nucleic acid sequencing using two or three different luminescent colors to identify four different types of nucleotides), provided herein is a composition a polymerase which is not detectably labeled and a single type of nucleotide, wherein the nucleotide is selected such that the type of nucleotide corresponds to a “dark” cycle (absence of a signal). In some embodiments, the composition comprises a buffer comprising a non-catalytic metal ion. In some embodiments, the non-catalytic metal ion is calcium, strontium, tin, or nickel. In some embodiments, the buffer does not comprise a catalytic metal ion cofactor of the polymerase. In some embodiments, the buffer does not comprise magnesium or manganese.

### Polymerase-Luciferase Fusion Proteins

“Luciferase” or “Luciferases” as used herein refer class of oxidative enzymes that produce bioluminescence. The non-secretory form of luciferases for use in the compositions and methods described herein lack the N-terminal secretory sequence but produce bioluminescence. In some embodiments, the luciferase is obtained from copepods, deep sea shrimp or homologs or orthologs thereof or mutants or derivatives thereof. In some embodiments, the copepods are any one or more of Gaussia princeps, Pleuromamma abdominalis, Metridia pacifica, Metridia curticauda, Metridia asymmetrica, Metridia okhotensis, Metridia longa, Lucicutia ovaliformis, Heterorhabdus tanneri, and Pleuromamma scutullata. In exemplary embodiments, the luciferases are any one or more of GLuc, NanoLuc (NLuc), MLuc7, HtLuc, LoLuc, PaLuc1, PaLuc2, MpLuc1, McLuc1, MaLuc1, MoLuc1, MoLuc2, MLuc39, PsLuc1, LocLuc1-3, HtLuc2 Renilla, TurboLuc16 (TLuc) or homologs or orthologs thereof or mutants or functional derivatives thereof. In some embodiments, the luciferase is a firefly or beetle luciferase. In various embodiments, mutants or functional derivatives of the luciferases retain at least 100%, 90%, 80%, 70%, 60% or 50% of the bioluminescence activity from which the mutant or function derivative is derived.

In some embodiments, the different types of luciferase are selected from the group consisting of a red-emitting luciferase, a yellow-emitting luciferase, a blue-emitting luciferase, and a green-emitting luciferase. Examples of luciferases, their emission spectra and colors, and their substrates have been described extensively, for example, in U.S. Pat. No. 11,768,203. In some embodiments, the yellow-emitting luciferase is a firefly luciferase or a derivative thereof. In some embodiments, the blue-emitting luciferase is an Amydetes viviani firefly luciferase or a derivative thereof. In some embodiments, the green-emitting luciferase is a P. vivianii railroad worm luciferase or a derivative thereof. In some embodiments, the red-emitting luciferase is a Phrixothrix hirtus railroad worm luciferase (PxRE) or a derivative thereof, or a luciferase derived from Photinus pyralis, Luciola cruciata, L. mingrelica or Pyrophorus plagiophtalamus that has been engineered to emit red light. In some embodiments, the far-red-emitting luciferase is a Phrixothrix hirtus railroad worm luciferase (PxRE) luciferase comprising an R215K mutation (PxRE-R215K), optionally wherein the PxRE-R215K luciferase is incubated with a 6′-(1-pyrrolidinyl)luciferin substrate.

In some embodiments, the luciferase is a copoped luciferase from a species selected from the group consisting of any one or more of Gaussia princeps, Pleuromamma abdominalis, Metridia pacifica, Metridia curticauda, Metridia asymmetrica, Metridia okhotensis, Metridia longa, Lucicutia ovaliformis, Heterorhabdus tanneri, and Pleuromamma scutullata.

In some embodiments, the luciferase is any one or more of GLuc, NanoLuc (NLuc), MLuc7, HtLuc, LoLuc, PaLuc1, PaLuc2, MpLuc1, McLuc1, MaLuc1, MoLuc1, MoLuc2, MLuc39, PsLuc1, LocLuc1-3, HtLuc2 Renilla, TurboLuc16 (TLuc) or homologs or orthologs thereof or mutants or functional derivatives thereof.

In some embodiments, the luminescent signal is assayed by exposing the biological sample a luciferase-specific substrate. In one embodiment, the luciferase-specific substrate is coelentrazine or a derivative thereof. In another embodiment, the luciferase-specific substrate is imidazopyrazinone substrate (furimazine) or a derivative thereof. In some embodiments, the substrate is luciferin.

In some embodiments, the luciferase is any one or more of GLuc, NanoLuc (NLuc), MLuc7, HtLuc, LoLuc, PaLuc1, PaLuc2, MpLuc1, McLuc1, MaLuc1, MoLuc1, MoLuc2, MLuc39, PsLuc1, LocLuc1-3, HtLuc2 Renilla, TurboLuc16 (TLuc) or homologs or orthologs thereof or mutants or derivatives thereof.

In some embodiments, at least one type of luciferase is GLuc (Gaussia princeps Luc). In some embodiments, at least one type of luciferase is NLuc (NanoLuc). In some embodiments, at least one type of luciferase is TLuc (TurboLuc16). In some embodiments, at least one type of luciferase is MLuc7-(Metrida longa) Luc M43L/M110L variant. In some embodiments, at least one type of luciferase is LoLuc (Lucicutia ovaliformis Luc). In some embodiments, at least one type of luciferase is HtLuc (Heterorhabdus. tanneri Luc). In some embodiments, at least one type of luciferase is PaLuc1 (Pleuromamma abdominalis Luc). In some embodiments, at least one type of luciferase is PaLuc2 (Pleuromamma abdominalis Luc2). In some embodiments, at least one type of luciferase is MpLuc1 (Metridia pacifica Luc1). In some embodiments, at least one type of luciferase is McLuc1 (Metridia curticauda Luc1). In some embodiments, at least one type of luciferase is MaLuc1 (Metridia asymmetrica Luc1). In some embodiments, at least one type of luciferase is MoLuc1 (Metridia okhotensis Luc1). In some embodiments, at least one type of luciferase is MoLuc2 (Metridia okhotensis Luc2). In some embodiments, at least one type of luciferase is MLuc39 (Metridia longa Luc39). In some embodiments, at least one type of luciferase is PsLuc1 (Pleuromamma scutullata Luc1). In some embodiments, at least one type of luciferase is LoLuc1-3 (Lucicutia ovaliformis Luc1-3). In some embodiments, at least one type of luciferase is HtLuc2 (Heterorhabdus tanneri Luc2). In some embodiments, at least one type of luciferase is Renilla Luc.

In some embodiments, the luciferase is fused to the N-terminus of the polymerase. In some embodiments the luciferase is fused to the C-terminus of the polymerase. In some embodiments the luciferase is fused to the N-terminus of the polymerase via a linker. In some embodiments, the luciferase is fused to the C-terminus of the polymerase via a linker.

Examples of polymerases that are used for performing the disclosed methods include, but are not limited to, DNA polymerases (e.g., Taq DNA polymerase), RNA polymerases, and/or reverse transcriptases.

In some embodiments, the polymerase of the polymerase conjugate is a DNA polymerase. Examples of DNA polymerases include Taq polymerase, 9° N-7 DNA polymerase (or variants thereof, for example, D141A/E143A/A485L), phi29 (φ29) polymerase, Klenow fragment, Bacillus stearothermophilus DNA polymerase (BST), T4 DNA polymerase, T7 DNA polymerase, and DNA polymerase I. In some embodiments, DNA polymerases that have been engineered or mutated to have desirable characteristics are employed. In some embodiments, the polymerase is phi29 DNA polymerase. In some embodiments, the polymerase of the polymerase conjugate is a DNA polymerase and the template nucleic acid molecule includes DNA. In some embodiments, the polymerase of the polymerase conjugate is a DNA polymerase and the nucleotide molecules include deoxyribonucleotides.

In some embodiments, the DNA polymerase is Taq polymerase or a functional variant thereof. Taq polymerase is a heat stable polymerase from Thermus aquaticus. An example Taq polymerase sequence is:

(SEQ ID NO: 1)
GMLPLFEPKGRVLLVDGHHLAYRTFHALKGLTTSRGEPVQAVYGFAKSL
LKALKEDGDAVIVVFDAKAPSFRHEAYGGYKAGRAPTPEDFPRQLALIK
ELVDLLGLARLEVPGYEADDVLASLAKKAEKEGYEVRILTADKDLYQLL
SDRIHVLHPEGYLITPAWLWEKYGLRPDQWADYRALTGDESDNLPGVKG
IGEKTARKLLEEWGSLEALLKNLDRLKPAIREKILAHMDDLKLSWDLAK
VRTDLPLEVDFAKRREPDRERLXAFLERLEFGSLLHEFGLLESPKXLXE
APWPPPERAFVP.

In some embodiments, the DNA polymerase is phi29 DNA polymerase or a functional variant thereof. The DNA polymerase of phi29 (a phage of Bacillus subtilis) has high processivity and fidelity. An example phi29 DNA polymerase sequence is:

(SEQ ID NO: 2)
MKHMPRKMYSCDFETTTKVEDCRVWAYGYMNIEDHSEYKIGNSLDEFMA
WVLKVQADLYFHNLKFDGAFIINWLERNGFKWSADGLPNTYNTIISRMG
QWYMIDICLGYKGKRKIHTVIYDSLKKLPFPVKKIAKDFKLTVLKGDID
YHKERPVGYKITPEEYAYIKNDIQIIAEALLIQFKQGLDRMTAGSDSLK
GFKDIITTKKFKKVFPTLSLGLDKEVRYAYRGGFTWLNDRFKEKEIGEG
MVFDVNSLYPAQMYSRLLPYGEPIVFEGKYVWDEDYPLHIQHIRCEFEL
KEGYIPTIQIKRSRFYKGNEYLKSSGGEIADLWLSNVDLELMKEHYDLY
NVEYISGLKFKATTGLFKDFIDKWTYIKTTSEGAIKQLAKLMLNSLYGK
FASNPDVTGKVPYLKENGALGFRLGEEETKDPVYTPMGVFITAWARYTT
ITAAQACYDRIIYCDTDSIHLTGTEIPDVIKDIVDPKKLGYWAHESTFK
RAKYLRQKTYIQDIYMKEVDGKLVEGSPDDYTDIKFSVKCAGMTDKIKK
EVTFENFKVGFSRKMKPKPVQVPGGVVLVDDTFTIK.

In some embodiments, the DNA polymerase is a 9° N-7 DNA polymerase or a functional variant thereof (e.g., D141A/E143A/A485L). 9° N-7 is a strain of Thermococcus sp. An example of a 9° N-7 DNA polymerase sequence is:

(SEQ ID NO: 3)
MILDTDYITENGKPVIRVFKKENGEFKIEYDRTFEPYFYALLKDDSAIE
DVKKVTAKRHGTVVKVKRAEKVQKKFLGRPIEVWKLYFNHPQDVPAIRD
RIRAHPAVVDIYEYDIPFAKRYLIDKGLIPMEGDEELTMLAFDIETLYH
EGEEFGTGPILMISYADGSEARVITWKKIDLPYVDVVSTEKEMIKRFLR
VVREKDPDVLITYNGDNFDFAYLKKRCEELGIKFTLGRDGSEPKIQRMG
DRFAVEVKGRIHFDLYPVIRRTINLPTYTLEAVYEAVFGKPKEKVYAEE
IAQAWESGEGLERVARYSMEDAKVTYELGREFFPMEAQLSRLIGQSLWD
VSRSSTGNLVEWFLLRKAYKRNELAPNKPDERELARRRGGYAGGYVKEP
ERGLWDNIVYLDFRSLYPSIIITHNVSPDTLNREGCKEYDVAPEVGHKF
CKDFPGFIPSLLGDLLEERQKIKRKMKATVDPLEKKLLDYRQRAIKILA
NSFYGYYGYAKARWYCKECAESVTAWGREYIEMVIRELEEKFGFKVLYA
DTDGLHATIPGADAETVKKKAKEFLKYINPKLPGLLELEYEGFYVRGFF
VTKKKYAVIDEEGKITTRGLEIVRRDWSEIAKETQARVLEAILKHGDVE
EAVRIVKEVTEKLSKYEVPPEKLVIHEQITRDLRDYKATGPHVAVAKRL
AARGVKIRPGTVISYIVLKGSGRIGDRAIPADEFDPTKHRYDAEYYIEN
QVLPAVERILKAFGYRKEDLRYQKTKQVGLGAWLKVKGKK.

In some embodiments, the DNA polymerase is DNA polymerase I or a functional fragment thereof (e.g., a Klenow fragment). Klenow fragment is an exonuclease deficient fragment of DNA polymerase I. An example of DNA polymerase I sequence is: MVQIPQNPLILVDGSSYLYRAYHAFPPLTNSAGEPTGAMYGVLNMLRSLIMQYKPTHAAV VFDAKGKTFRDELFEHYKSHRPPMPDDLRAQIEPLHAMVKAMGLPLLAVSGVEADDVIGT LAREAEKAGRPVLISTGDKDMAQLVTPNITLINTMTNTILGPEEVVNKYGVPPELIIDFLALM GDSSDNIPGVPGVGEKTAQALLQGLGGLDTLYAEPEKIAGLSFRGAKTMAAKLEQNKEVA YLSYQLATIKTDVELELTCEQLEVQQPAAEELLGLFKKYEFKRWTADVEAGKWLQAKGAK PAAKPQETSVADEAPEVTATVISYDNYVTILDEETLKAWIAKLEKAPVFAFDTETDSLDNIS ANLVGLSFAIEPGVAAYIPVAHDYLDAPDQISRERALELLKPLLEDEKALKVGQNLKYDRGI LANYGIELRGIAFDTMLESYILNSVAGRHDMDSLAERWLKHKTITFEEIAGKGKNQLTFNQI ALEEAGRYAAEDADVTLQLHLKMWPDLQKHKGPLNVFENIEMPLVPVLSRIERNGVKIDP KVLHNHSEELTLRLAELEKKAHEIAGEEFNLSSTKQLQTILFEKQGIKPLKKTPGGAPSTSEE VLEELALDYPLPKVILEYRGLAKLKSTYTDKLPLMINPKTGRVHTSYHQAVTATGRLSSTDP NLQNIPVRNEEGRRIRQAFIAPEDYVIVSADYSQIELRIMAHLSRDKGLLTAFAEGKDIHRAT AAEVFGLPLETVTSEQRRSAKAINFGLIYGMSAFGLARQLNIPRKEAQKYMDLYFERYPGV LEYMERTRAQAKEQGYVETLDGRRLYLPDIKSSNGARRAAAERAAINAPMQGTAADIIKR AMIAVDAWLQAEQPRVRMIMQVHDELVFEVHKDDVDAVAKQIHQLMENCTRLDVPLLVE VGSGENWDQAH (SEQ ID NO: 4). In some embodiments, a Klenow fragment includes positions 324-928 with respect to SEQ ID NO: 4.

In some embodiments, the polymerase of the polymerase conjugate is a reverse transcriptase. Reverse transcriptases typically have RNA-dependent DNA polymerase activity and DNA-dependent DNA polymerase activity. Examples of reverse transcriptases include Moloney murine leukemia virus (MMLV) reverse transcriptase, HIV-1 reverse transcriptase, and avian myeloblastosis virus (AMV) reverse transcriptase. In some embodiments, the reverse transcriptase lacks (e.g., is mutated to lack) ribonuclease activity. In some embodiments, ribonuclease activity degrade template particularly during longer incubation times such as when reverse transcribing longer cDNAs. In some embodiments, the polymerase of the polymerase conjugate is a reverse transcriptase and the template nucleic acid molecule is an RNA molecule. In some embodiments, the polymerase of the polymerase conjugate is a reverse transcriptase and the nucleotide molecules include deoxyribonucleotide molecules.

In some embodiments, the reverse transcriptase is an MMLV reverse transcriptase or a functional variant thereof. An example of an MMLV reverse transcriptase sequence is: AFPLERPDWDYTTQAGRNHLVHYRQLLLAGLQNAGRSPTNLAKVKGITQGPNESPSAFLER LKEAYRRYTPYDPEDPGQETNVSMSFIWQSAPDIGRKLGRLEDLKSKTLGDLVREAEKIFN KRETPEEREERIRRETEEKEERRRTVDEQKEKERDRRRHREMSKLLATVVIGQEQDRQEGE RKRPQLDKDQCAYCKEKGHWAKDCPKKPRGPRGPRPQTSLLTLGDXGGQGQDPPPEPRIT LKVGGQPVTFLVDTGAQHSVLTQNPGPLSDKSAWVQGATGGKRYRWTTDRKVHLATGK VTHSFLHVPDCPYPLLGRDLLTKLKAQIHFEGSGAQVVGPMGQPLQVLTLNIEDEYRLHETS KEPDVSLGFTWLSDFPQAWAESGGMGLAVRQAPLIIPLKATSTPVSIKQYPMSQEARLGIKP HIQRLLDQGILVPCQSPWNTPLLPVKKPGTNDYRPVQDLREVNKRVEDIHPTVPNPYNLLSG LPPSHQWYTVLDLKDAFFCLRLHPTSQPLFAFEWRDPEMGISGQLTWTRLPQGFKNSPTLF DEALHRDLADFR (SEQ ID NO: 5). Residues 431-560 of SEQ ID NO: 5 provide reverse transcriptase activity.

In some embodiments, the reverse transcriptase is an HIV-1 reverse transcriptase or a functional variant thereof. An example of an HIV-1 reverse transcriptase sequence is:

(SEQ ID NO: 6)
PISPIEPVPVKLKPGMDGPKVKQWPLTEEKIKALVEICTEMEKEGKISK
IGPENPYNTPVFAIKKKDSTRWRKLVDFRELNKRTQDFWEVQLGIPHPA
GLKKKRSVTVLDVGDAYFSVPLDKEFRKYTAFTIPSINNETPGIRYQYN
VLPQGWKGSPAIFQSSMTKILEPFRKQNPDIVIYQYMDDLYVGSDLEIG
QHRTKIEELRQHLLKWGFTTPDKKHQKEPPFLWMGYEHHPDKWTVQPIV
LPEKDSWTVNDIQK.

In some embodiments, the polymerase is selected from Taq polymerase, 9° N-7 DNA polymerase or a functional variant thereof (e.g., D141A/E143A/A485L), and a Klenow fragment of DNA polymerase I. In some embodiments, the polymerase is not labeled with a detectable label.

In some embodiments, the luciferase is attached to the polymerase via a flexible linker (e.g., an amino acid linker). In some embodiments, the linker comprises a glycine and/or serine linker. In some embodiments, is at least 1, 2, 3, 4, 5, 6, 7, 8, 9, 10, 15, 20, 25, 30, 40, or 50 amino acids in length. In some embodiments, the linker is between 1 and 50 amino acids in length.

### III. Nucleic Acid Sequencing Using Polymerase-Luciferase Fusion Proteins

The sequencing methods described herein are useful for multi-cycle sequencing approaches where nucleotides of nucleotide strand are “interrogated” by binding to a complementary nucleotide. For example, the sequencing methods described herein are applicable to both in situ sequencing applications (e.g., in situ sequencing of endogenous nucleic acid sequences and/or target-specific barcode sequences associated with target analytes of interest that are distributed within a cell or tissue sample) and to more conventional “sequencing in a flow cell” applications (e.g., sequencing of endogenous nucleic acid sequences extracted from a cell or tissue sample). The in situ and flow cell sequencing approaches differ in terms of the sample preparation steps required, as described elsewhere herein, but can share common features in terms of the cyclic series of steps performed to identify nucleotides base-by-base in a template nucleic acid sequence (e.g., a target analyte sequence and/or an associated target-specific barcode sequence).

In some embodiments, the sequencing methods described herein comprise contacting a template nucleic acid molecule with a sequencing primer designed to hybridize to a portion of the template nucleic acid molecule. In some embodiments, the sequencing primer comprises a free 3′-hydroxyl group at its 3′ terminus. In some embodiments, a nucleotide that is contacted with the biological sample together with a polymerase or a polymerase-luciferase fusion protein is not incorporated into the sequencing primer by the polymerase or polymerase-luciferase fusion protein in the presence of a buffer that lacks a catalytic metal ion cofactor of the polymerase and/or that comprises a non-catalytic metal ion such as calcium, strontium, tin, or nickel. In some embodiments, the complementary nucleotide is incorporated into the priming strand (e.g., the sequencing primer or an extended sequencing primer) upon contacting the biological sample with a buffer comprising a catalytic metal ion cofactor of the polymerase. In some embodiments, the nucleotide does not comprise a reversible terminator. In some embodiments, the sample is washed prior to contacting the sample with a buffer comprising a catalytic metal ion cofactor of the polymerase to remove unbound polymerases and nucleotides, such that only a single nucleotide is present and is incorporated at each primed template nucleic acid sequence. In some embodiments, the biological sample is washed to remove the catalytic metal ion cofactor before a subsequent cycle of base-by-base sequencing.

### Template Nucleic Acid Molecules

In some embodiments, the template nucleic acid molecule is a rolling circle amplification product comprising a plurality of copies of a sequence of interest (e.g., a barcode sequence or a marker sequence that identifies a nucleic acid analyte) adjacent to a sequencing primer-binding sequence. During in situ sequencing, sequencing primers are bound adjacent to the copies of sequences of interest and extended to sequence a portion of the sequence of interest in a nucleic acid sequencing reaction. In some embodiments, a common primer sequence is present in each of a plurality of sequences of interest, such that a common sequencing primer can be used to sequence the different sequences of interest. In some embodiments, a plurality of sequences of interest comprise a common sequencing primer-binding region and different barcode or marker sequences 5′ of the sequencing primer-binding region. In some embodiments, the marker sequence is a sequence of the target nucleic acid molecule that is targeted by a circularizable probe or probe set. In some embodiments, the complement of the marker sequence is incorporated into a circularizable probe or probe set in a gap-fill reaction (e.g., splint-mediated gap-fill or gap-fill by extension using a polymerase) using the target nucleic acid molecule as a template.

In some aspects, the methods comprising luminescence detection for sequencing provided herein advantageously avoid problems such as photobleaching and autofluorescence that occur with fluorescent imaging techniques requiring excitation, particularly in cell and tissue samples comprising autofluorescent moieties. In some embodiments, the method comprises sequencing a sequence of interest in an RCP comprising no more than 5, 10, 15, or 20 copies of the sequence of interest.

In some embodiments, the template nucleic acid molecule includes a target analyte nucleic acid molecule (e.g., a DNA molecule, an RNA molecule, or an mRNA molecule). In some embodiments, the template nucleic acid includes a reporter oligonucleotide, such as a barcode.

In some embodiments, the template nucleic acid molecule is a DNA molecule. Examples of DNA template nucleic acid molecules include DNA molecules such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. In some embodiments, the DNA molecule is copied from another nucleic acid molecule (e.g., DNA or RNA such as mRNA).

In some embodiments, the template nucleic acid molecule is an RNA molecule. Examples of RNA template nucleic acid molecules include RNA molecules such as various types of coding and non-coding RNA. Examples of the different types of RNA molecules include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. In some embodiments, the RNA template nucleic acid molecule is copied from another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. In some embodiments, the RNA is small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). In some embodiments, the RNA is double-stranded RNA or single-stranded RNA. In some embodiments, the RNA is circular RNA. In some embodiments, the RNA is a bacterial rRNA (e.g., 16s rRNA or 23s rRNA).

In some embodiments, the template nucleic acid comprises a nucleic acid analyte derived from a biological sample and/or a reporter oligonucleotide or nucleic acid marker associated with an analyte from a biological sample. Such analytes can be or derived from any biological sample. In some embodiments, the template nucleic acid comprises a nucleic acid analyte and/or a reporter oligonucleotide or nucleic acid marker associated with an analyte present in a biological sample, and the template nucleic acid molecule is sequenced at a location in the biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, in some embodiments, a biological sample is obtained from a prokaryote such as a bacterium, an archaea, a virus, or a viroid. In some embodiments, a biological sample is obtained from non-mammalian organisms (e.g., a plant, an insect, an arachnid, a nematode, a fungus, or an amphibian). In some embodiments, a biological sample is obtained from a eukaryote, such as a tissue sample, a patient derived organoid (PDO) or patient derived xenograft (PDX). In some embodiments, a biological sample from an organism comprises one or more other organisms or components therefrom. For example, a mammalian tissue section may comprise a prion, a viroid, a virus, a bacterium, a fungus, or components from other organisms, in addition to mammalian cells and non-cellular tissue components. In some embodiments, subjects from which biological samples are obtained are healthy or asymptomatic individuals, individuals that have or are suspected of having a disease (e.g., a patient with a disease such as cancer) or a pre-disposition to a disease, and/or individuals in need of therapy or suspected of needing therapy.

In some embodiments, a template nucleic acid includes a reporter oligonucleotide or marker associated with the presence of an analyte (e.g., an endogenous analyte) in a sample. Such analytes may include nucleic acid analytes and/or non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Examples of analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In some embodiments, a template nucleic acid molecule may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In some embodiments, methods and compositions disclosed herein are used to analyze any number of template nucleic acid molecules (e.g., nucleic acid analytes and/or analyte-associated barcode sequences) or fragments thereof. For example, in some embodiments, the number of analytes that are analyzed is at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of a sample (e.g., a cell sample or tissue sample) or tethered within individual features on a substrate (e.g., a flow cell surface).

### Workflows for Performing Sequencing

The disclosed sequencing methods may be applied to both in situ sequencing and flow cell sequencing applications, where the sequencing reactions are substituted for the stepwise nucleotide incorporation reactions used to probe a template nucleic acid sequence in, e.g., a conventional sequencing-by-synthesis (SBS) method.

In the case of in situ sequencing, in some embodiments, the disclosed methods comprise performing all or a subset of the following steps (in addition to a cyclic series of base-by-base sequencing reactions):

(i) preparing the biological sample (e.g., by fixing, sectioning, embedding, and/or clearing a cell or tissue sample, as described elsewhere herein).

(ii) contacting target analytes (e.g., target nucleic acid analytes and/or protein analytes) within the prepared sample with target-specific probes, as described elsewhere herein. In some embodiments, the target-specific probes comprise, e.g., target-specific linear and/or circularizable nucleic acid probes (e.g., padlock probes) designed to hybridize directly or indirectly to specific target nucleic acid analytes. In some embodiments, the target-specific linear and/or circularizable nucleic acid probes comprise primer binding sites and/or target-specific barcode (or identifier) sequences. In some embodiments, the target-specific probes comprise, e.g., target-specific antibodies designed to bind to specific target protein analytes, where the antibodies are conjugated to nucleic acid sequences. In some embodiments, the conjugated nucleic acid sequences comprise primer binding sites and/or target-specific barcode (or identifier) sequences.

(iii) optionally performing a reverse transcription reaction (e.g., if the probed target nucleic acid analytes comprise RNA molecules) to create cDNA copies of RNA target molecules.

(iv) optionally amplifying the probed target analyte molecules and/or their associated target-specific barcode sequences (e.g., using rolling circle amplification (RCA) in the case that target-specific circularizable probes were used to probe target analyte molecules and/or associated barcode sequences).

(v) contacting the optionally amplified target nucleic acid analytes and/or associated target-specific barcode sequences with sequencing primers designed to hybridize directly or indirectly to the target nucleic acid analytes and/or their associated target-specific barcode sequences.

In the case of flow cell sequencing, in some embodiments, the disclosed methods comprise performing all or a subset of the following steps (in addition to a cyclic series of base-by-base sequencing reactions):

(i) extraction and purification of nucleic acid molecules (e.g., endogenous nucleic acid sequences) from a biological sample, as described elsewhere herein.

(ii) preparation of a sequencing library comprising template nucleic acid molecules (e.g., the endogenous nucleic acid sequences or fragments thereof) that have been end-repaired and ligated to adapter sequences, as described elsewhere herein.

(iii) optionally performing nucleic acid amplification of all or a portion of the sequencing library, as described elsewhere herein.

(iv) immobilizing the template nucleic acid molecules (e.g., denatured, single-stranded template nucleic acid molecules) from the sequencing library on an inner surface of a flow cell using capture probes (e.g., complementary adapter sequences) that have been tethered to the flow cell surface.

(v) performing clonal amplification of the immobilized template nucleic acid molecules to create clusters comprising, e.g., thousands or tens of thousands of copies of the template nucleic acid molecule immobilized at each of a plurality of locations on the flow cells surface.

(vi) contacting the template nucleic acid molecules in each clonally-amplified cluster with sequencing primers designed to hybridize to, e.g., the adapter sequences ligated to the template nucleic acid molecules.

In the case of either in situ sequencing or flow cell sequencing, in some embodiments, the disclosed methods comprise: performing a cyclic series of base-by-base sequencing reactions, where each sequencing cycle comprises contacting the sample with a composition comprising polymerase-luciferase fusion protein and a single type of nucleotide under conditions that promote formation of a stalled ternary complex. In some embodiments, the disclosed methods comprise: performing a cyclic series of base-by-base sequencing reactions, where each sequencing cycle comprises contacting the sample with a composition comprising polymerase-luciferase fusion protein and a single type of nucleotide in the presence of a non-catalytic dication (e.g., calcium, strontium, nickel, or tin) and in the absence of a catalytic metal cation cofactor of the polymerase. In some embodiments, each cycle comprises contacting the sample with a substrate of the luciferase. In some embodiments, each cycle comprises detecting a luminescent signal in the presence of the substrate, thereby identifying the complementary base in the template nucleic acid. In some embodiments, after identifying the complementary base in the template nucleic acid, the method comprises contacting the sample with a buffer comprising a metal cation cofactor of the polymerase, thereby allowing incorporation of the nucleotide into the priming strand. In some embodiments, the method comprises replacing the buffer on the sample with a buffer comprising the metal cation cofactor of the polymerase.

In some embodiments, the disclosed methods further comprise processing optical signals (e.g., luminescent signals) detected in images (e.g., luminescent images) acquired during the cyclic series of base-by-base sequencing reactions to detect the presence or absence of complementary single nucleotides in complex with the template nucleic acid and a polymerase-luciferase fusion protein in each sequencing cycle at the locations of each of a plurality of template nucleic acid molecules (i.e., the locations corresponding to each of a plurality of target analyte molecules and/or their associated target-specific barcode sequences), thereby enabling inference of the nucleotide sequence of the plurality of template nucleic acid molecules (e.g., the plurality of target analyte molecules and/or associated target-specific barcode sequences).

In some embodiments, the cyclic series of base-by-base sequencing reactions comprises performing at least 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, or more than 50 cycles of the base-by-base sequencing reaction.

In some embodiments, each cycle of base-by-base sequencing further comprises a first wash step following the contacting step to remove unbound polymerase-luciferase fusion proteins and nucleotides. In some embodiments, the first wash step comprises, for example, use of the same buffer used for contacting the primed template nucleic acid with the polymerase-luciferase fusion protein and corresponding nucleotide (but without the polymerase-luciferase fusion protein and nucleotide or nucleotide composition). In some embodiments, the first wash buffer may not include KCl and/or may include little to no DMSO. In some embodiments, the first wash buffer does not comprise a catalytic ion cofactor of the polymerase. In some embodiments, the first wash buffer is similar to those used for wash buffers as used in wash steps of a Western blot (e.g., a wash buffer added in a Western blot after binding a primary antibody but washing prior to incubation with a secondary antibody, such as PBST). PBST is a phosphate-buffered saline with a low-concentration of detergent, such as 0.05% to 0.1% Tween.

In some embodiments, the detection step comprises the use of an optical imaging technique (e.g., a fluorescence imaging technique) and real time or post-processing measurement of optical signals (e.g., luminescent signals or the absence thereof) associated with the presence of a specific polymerase-luciferase fusion protein at a plurality of locations corresponding to a plurality of target analytes distributed throughout the biological sample or tethered to specific locations on a substrate surface (e.g., a flow cell surface).

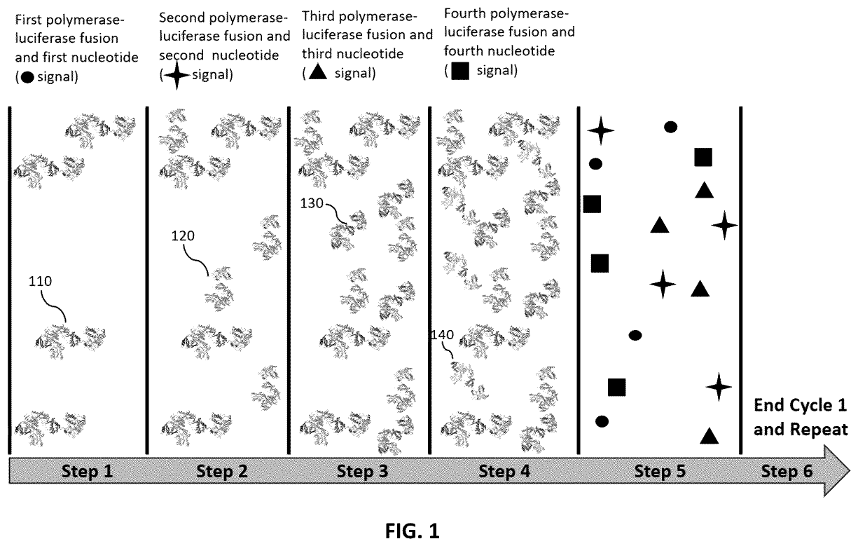

FIG. 1 provides a non-limiting example of a workflow for a process for sequencing a template nucleic acid molecule in accordance with one implementation of the methods described herein. In some embodiments, the method comprises sequentially contacting the sample with a first type of polymerase-luciferase fusion 110 and a first type of nucleotide (Step 1 in FIG. 1), a second type of polymerase-luciferase fusion 120 and a second type of nucleotide (Step 2 in FIG. 1), a third type of polymerase-luciferase fusion 130 and a third type of nucleotide (Step 3 in FIG. 1), and a fourth type of polymerase-luciferase fusion 140 and a fourth type of nucleotide (Step 4 in FIG. 1). In some embodiments, the method comprises washing the sample between each of Steps 1-4 to remove unbound nucleotides and polymerase-luciferase fusion proteins. FIG. 1 schematically illustrates a view from above a biological sample, wherein different types of polymerase-luciferase fusion proteins and corresponding nucleotides form stalled ternary complexes at different locations, because primed template nucleic acid molecules at the different locations have different nucleobases present in the template nucleic acid sequence. In some embodiments, after flowing in all of the different types of luciferases, the sample is contacted with the substrate(s) of the different types of luciferases and imaged to detect luminescent signals corresponding to the stalled ternary complexes comprising different types of nucleotides at their locations in the biological sample (Step 5 in FIG. 1). In some embodiments, after detecting the luminescent signals, the method comprises contacting the biological sample with a buffer comprising a catalytic ion cofactor of the polymerase (e.g., magnesium) to allow incorporation of the nucleotides and release of the ternary complexes. In some embodiments, some steps are combined, the order of some steps are changed, and/or some steps are omitted. In some embodiments, additional steps are performed in combination with the steps shown in FIG. 1. Accordingly, the steps illustrated (and described in greater detail below) for the method are exemplary by nature, and as such, should not be viewed as limiting.

In some embodiments, the template nucleic acid molecule comprises an endogenous nucleic acid molecule (e.g., a DNA molecule, an RNA molecule, or an mRNA molecule) that has been reverse transcribed, amplified, and/or extracted from a biological sample (e.g., a cell sample or tissue sample).

In some embodiments, the template nucleic acid molecule comprises a barcode sequence (e.g., a nucleic acid barcode sequence) associated with a target analyte of interest (e.g., using the barcoding methods described elsewhere herein) that has been reverse transcribed, amplified, and/or extracted from a biological sample (e.g., a cell sample or tissue sample).

In some embodiments, the method further comprises hybridizing a circularizable probe to a target analyte (or to a labeling agent bound to the target analyte), ligating the circularizable probe to form a circularized probe, and performing rolling circle amplification of the circularized probe to generate the template nucleic acid molecule. In some embodiments, for example, the circularizable probe is a padlock probe sequence.

In some embodiments, the template nucleic acid molecule to be sequenced is attached to a solid support, e.g., a sequencing flow cell.

In some embodiments, the template nucleic acid molecule is sequenced in situ in a cell sample or tissue sample. In some embodiments, the cell sample comprises a layer of cells deposited on a surface.

In some embodiments, the polymerase comprises, e.g., Taq polymerase, Therminator™ DNA polymerase, a Klenow fragment of DNA polymerase I, or any combination thereof. In some embodiments, the polymerase is not labeled with a detectable label.

In some embodiments, each cycle of a cyclic series of base-by-base sequencing reactions performed as part of the disclosed methods for in situ or flow cell sequencing comprises contacting priming strands bound to template nucleic acid molecules with a composition comprising a polymerase-luciferase fusion protein and a single type of nucleotide. In some embodiments, each cycle of a cyclic series of base-by-base sequencing reactions comprises sequentially contacting the priming strand bound to a template nucleic acid molecule with a first composition comprising a first type of polymerase-luciferase fusion protein and a single first type of nucleotide, and then with a one or more additional compositions individually comprising a different type of polymerase-luciferase fusion protein and a different single type of nucleotide. In some embodiments, the single type of nucleotide in each composition comprises a same type of nucleobase (e.g., selected from A, T, U, C, and/or G). In some embodiments, each different type of nucleotide comprises a different nucleobase.

In some instances, each cycle of a cyclic series of base-by-base sequencing reactions performed as part of the disclosed methods for sequencing (e.g., in situ or flow cell sequencing) may comprise contacting priming strands bound to template nucleic acid molecules with at least one modified nucleotide molecule comprising the first functional group (e.g., a click chemistry reactive group, as described elsewhere herein). In some instances, each cycle of a cyclic series of base-by-base sequencing reactions may comprise contacting priming strand bound to a template nucleic acid molecule with a plurality of modified nucleotide molecules (e.g., 2, 3, or 4 modified nucleotide molecules) comprising the first functional group.

Such modified nucleotide molecules may be modified versions of any of a variety of naturally-occurring nucleotides and/or functional analogs thereof (e.g., nucleotide analogs capable of hybridizing to a nucleic acid sequence in a sequence-specific/correctly base-paired manner) to probe a template nucleic acid sequence. Naturally-occurring nucleotides include deoxyribonucleotides (found in DNA) that comprise a deoxyribose sugar moiety, and ribonucleotides (found in RNA) that comprise a ribose sugar moiety. Naturally-occurring deoxyribonucleotides comprise a nucleobase (or “base”) selected from the group consisting of adenine (A), thymine (T), cytosine (C), or guanine (G). Naturally-occurring ribonucleotides comprise a nucleobase selected from the group consisting of uracil (U), adenine (A), cytosine (C), or guanine (G). In some instances, the nucleotides may be terminated (e.g., reversibly terminated). In some instances, the nucleotides may be conjugated to a detectable label, e.g., a fluorophore. In some instances, as noted above, the nucleotides may be conjugated to other moieties, e.g., reactive functional groups.

In some instances, the set of one or more modified nucleotide molecules contacted with the primed template nucleic acid molecule(s) in each cycle of a multicycle sequencing process may comprise modified versions of A, T, U, C, and/or G. In some instances, the set of one or more modified nucleotide molecules contacted with the primed template nucleic acid molecule(s) in each cycle of a multicycle sequencing process may comprise modified versions of A, T, C, and/or G.

In some instances, the set of one or more modified nucleotide molecules contacted with the primed template nucleic acid molecule(s) may be the same in each cycle of a multicycle sequencing process (e.g., the one or more modified nucleotide molecules may comprise the same selection of modified A, T, U, C, and/or G in each cycle). In some instances, the set of one or more modified nucleotide molecules contacted with the primed template nucleic acid molecule(s) may be different in different cycles of a multicycle sequencing process (e.g., the one or more modified nucleotide molecules may comprise a different selection of modified A, T, U, C, and/or G in different cycles).

In some instances, the set of one or more modified nucleotide molecules contacted with the primed template nucleic acid molecule(s) may be coupled to one or more detectable labels, where different modified nucleotide molecules (e.g., modified nucleotides comprising different nucleobases) are labeled with different detectable labels. In some instances, the one or more detectable label(s) may comprise one or more fluorophores. Examples of suitable detectable labels, e.g., fluorophores, are described elsewhere herein.

In some instances, all of the modified nucleotide molecules in the set of one or more modified nucleotide molecules (e.g., modified versions of A, T, U, C, and/or G) contacted with the primed template nucleic acid molecule(s) may be coupled to detectable labels (e.g., fluorophores). In some instances, at least one modified nucleotide molecule of the set of one or more modified nucleotide molecules contacted with the primed template nucleic acid molecules may not be coupled to a detectable label. In some instances, non-labeled modified nucleotide molecules may be used to implement different detection schemes (e.g., two color, three color, or four color detection schemes). In some instances, non-labeled modified nucleotide molecules may be used to implement the readout of target-specific barcode designs used to minimize optical crowding when performing in situ sequencing (see, e.g., PCT International Patent Application Publication Nos. WO 2022/060889, U.S. patent application Ser. No. 17/476,398, U.S. patent application Ser. No. 17/476,400, U.S. patent application Ser. No. 17/476,395, WO 2023/220300, U.S. patent application Ser. No. 18/196,333, U.S. patent application Ser. No. 18/589,354, and U.S. Pat. No. 12,270,074, each of which is incorporated herein by reference in its entirety).

In some instances, for example, the set of one or more modified nucleotide molecules may comprise a set of four different modified nucleotide molecules (e.g., selected from modified versions of A, T, U, C, and/or G), where each different modified nucleotide molecule (comprising a different nucleobase) is coupled to a different fluorophore.

In some instances, the set of one or more modified nucleotide molecules may comprise a set of four different modified nucleotide molecules (e.g., selected from modified versions of A, T, U, C, and/or G), where three of the four different modified nucleotide molecules (comprising different nucleobases) are coupled to different fluorophores and one of the four different modified nucleotide molecules is not conjugated to a fluorophore.

In some instances, the set of one or more modified nucleotide molecules may comprise a set of four different modified nucleotide molecules (e.g., selected from modified versions of A, T, U, C, and/or G), where two of the four different modified nucleotides (comprising different nucleobases) are coupled to different fluorophores, one of the four different modified nucleotide molecules is coupled to both of the two different fluorophores, and one of the four different modified nucleotide molecules is not conjugated to a fluorophore.

Methods for processing the series of optical signals detected over the course of performing a cyclic series of base-by-base sequencing reactions to identify a nucleotide sequence are described elsewhere herein.

### IV. Kits

In some embodiments, provided herein are kits and/or systems for sequencing nucleic acid molecules, including kits and/or systems for sequencing and analysis of target nucleic acids in a biological sample according to any of the methods described herein.

In some embodiments, provided herein is a kit comprising any of the composition comprising polymerase-luciferase fusion proteins described herein. In some embodiments, the kit further comprises any of the primers described herein. In some embodiments, provided herein is a system comprising the kit and a biological sample.

In some embodiments, provided herein is a kit for performing in situ sequencing comprising a composition comprising one or more polymerase-luciferase fusion proteins as described herein, and one or more further components for performing the in situ sequencing reaction. In some embodiments, the one or more further components include a polymerase, a primer, a support for a tissue or cell sample, or any combination thereof. In some embodiments, the kit further comprises any of the circular probes and/or circularizable probes or probe sets disclosed herein. In some embodiments, the kit comprises a polymerase for rolling circle amplification. In some embodiments, provided herein is a system comprising the kit and a biological sample.

In some embodiments, provided herein is a kit for flow cell sequencing comprising a composition comprising polymerase-luciferase fusion proteins as described herein, and one or more further components for performing the flow cell sequencing reaction. In some embodiments, the one or more further components include a polymerase, a primer, a flow cell, primers, adapters for sequencing library preparation, or any combination thereof. In some embodiments, provided herein is a system comprising the kit and a biological sample.

In some embodiments, provided herein is a kit for sequencing a template nucleic acid molecule comprising: a first type of polymerase-luciferase fusion protein and a first type of nucleotide, a second type of polymerase-luciferase fusion protein and a second type of nucleotide, wherein the first type of polymerase-luciferase fusion comprises a first type of luciferase that emits a first type of luminescent signal and the second type of polymerase-luciferase fusion comprises a second type of luciferase that emits a second type of luminescent signal. In some embodiments, provided herein is a system comprising the kit and a biological sample.

In some embodiments, (e.g., for 2-color sequencing chemistry), the kit comprises a composition comprising a mixture of the first type of polymerase-luciferase fusion protein and the second type of polymerase-luciferase fusion protein, and a third type of nucleotide. In some embodiments, the kit comprises a third type of nucleotide and instructions for contacting the biological sample with the third type of nucleotide together with a mixture of the first type of polymerase-luciferase fusion protein and the second type of polymerase-luciferase fusion protein (e.g., for 2-color sequencing chemistry). In some embodiments, (e.g., for 2-color sequencing chemistry), the kit comprises a fourth type of nucleotide and a polymerase. In some embodiments, the fourth type of nucleotide and the polymerase do not comprise fluorescent or luminescent labels. In some embodiments, provided herein is a system comprising the kit and a biological sample.

In some embodiments, (e.g., for 3-color sequencing chemistry), the kit comprises a third type of polymerase-luciferase fusion protein and a third type of nucleotide, wherein the third type of polymerase-luciferase fusion comprises a third type of luciferase that emits a third type of luminescent signal indicating formation of a ternary complex comprising the third type of nucleotide, and a fourth type of nucleotide and a polymerase. In some embodiments, the fourth type of nucleotide and the polymerase do not comprise fluorescent or luminescent labels. In some embodiments, provided herein is a system comprising the kit and a biological sample.

In some embodiments, (e.g., for 4-color sequencing chemistry), the kit comprises a third type of polymerase-luciferase fusion protein and a third type of nucleotide, wherein the third type of polymerase-luciferase fusion comprises a third type of luciferase that emits a third type of luminescent signal indicating formation of a ternary complex comprising the third type of nucleotide, and a fourth type of polymerase-luciferase fusion protein and a fourth type of nucleotide, wherein the fourth type of polymerase-luciferase fusion comprises a fourth type of luciferase that emits a fourth type of luminescent signal indicating formation of a ternary complex comprising the fourth type of nucleotide. In some embodiments, provided herein is a system comprising the kit and a biological sample.

In some embodiments of the kits provided herein, the first, second, third, and fourth types of nucleotides are A, T or U, C, and G. In some embodiments, the first, second, third, and fourth types of nucleotides are A, T, C, and G. In some embodiments, the first, second, third, and fourth types of nucleotides are A, U, C, and G. In some embodiments, the first type of nucleotide, second type of nucleotide, third type of nucleotide, and fourth type of nucleotide comprise different nucleobases selected from the group consisting of A, T, C, and G. In some embodiments, the first type of nucleotide and second type of nucleotide are not reversibly terminated nucleotides. In some embodiments, the first type of nucleotide, second type of nucleotide, and third type of nucleotide are not reversibly terminated nucleotides. In some embodiments, the first type of nucleotide, second type of nucleotide, third type of nucleotide, and fourth type are not reversibly terminated nucleotides. In some embodiments, the first type of nucleotide, second type of nucleotide, third type of nucleotide, and fourth type of nucleotide are reversibly terminated nucleotides. In some embodiments, first type of nucleotide, second type of nucleotide, third type of nucleotide, and fourth type of nucleotide are not detectably labeled (e.g., are not fluorescently labeled). In some embodiments, provided herein is a system comprising the kit and a biological sample.

In some embodiments of the kits provided herein, the first and second types of luciferase are different types of luciferase independently selected from the group consisting of a yellow-emitting luciferase, a green-emitting luciferase, a blue-emitting luciferase, a red-emitting luciferase, and a far-red-emitting luciferase. In some embodiments, the first, second, and third types of luciferase are different types of luciferase independently selected from the group consisting of a yellow-emitting luciferase, a green-emitting luciferase, a blue-emitting luciferase, a red-emitting luciferase, and a far-red-emitting luciferase. In some embodiments, the first, second, third, and fourth types of luciferase are different types of luciferase independently selected from the group consisting of a yellow-emitting luciferase, a green-emitting luciferase, a blue-emitting luciferase, a red-emitting luciferase, and a far-red-emitting luciferase. In some embodiments, the kit comprises one or more luciferase substrates. In some embodiments, provided herein is a system comprising the kit and a biological sample.

In some embodiments, the kit comprises one or more buffers, reagents for one or more buffers, or instructions for preparing one or more buffers for contacting the biological sample with a polymerase-luciferase fusion protein under ternary complex-stabilizing conditions. In some embodiments, the ternary complex-stabilizing conditions comprise presence of a non-catalytic metal ion. In some embodiments, the ternary complex-stabilizing conditions comprise absence of a catalytic metal ion cofactor of the polymerase. In some embodiments, the catalytic metal ion is magnesium or manganese. In some embodiments, the non-catalytic metal ion is calcium, strontium, tin, or nickel. In some embodiments, provided herein is a system comprising the kit and a biological sample.

In some embodiments, the kit comprises one or more wash buffers, reagents for one or more wash buffers, or instructions for preparing one or more wash buffers for washing the biological sample under ternary complex-stabilizing conditions. In some embodiments, the ternary complex-stabilizing conditions comprise presence of a non-catalytic metal ion. In some embodiments, the ternary complex-stabilizing conditions comprise absence of a catalytic metal ion cofactor of the polymerase. In some embodiments, the catalytic metal ion is magnesium or manganese. In some embodiments, the non-catalytic metal ion is calcium, strontium, tin, or nickel. In some embodiments, provided herein is a system comprising the kit and a biological sample.

In some embodiments, the kit further comprises one or more buffers, reagents for one or more buffers, or instructions for preparing one or more buffers that allow for incorporation of a nucleotide by a polymerase. In some embodiments, the one or more buffers that allow for incorporation of a nucleotide by a polymerase comprise a catalytic metal ion (e.g., magnesium or manganese). In some embodiments, provided herein is a system comprising the kit and a biological sample.

The various components of the kit may be present in separate containers or certain compatible components may be pre-combined into a single container. In some embodiments, the kits further contain instructions for using the components of the kit to practice the provided methods. In some embodiments, each composition comprising a type of polymerase-luciferase fusion protein is provided in separate containers. In some embodiments, each type of polymerase-luciferase fusion protein is provided in separate containers.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some embodiments, the kit also comprises any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers.

### V. Biological Samples & Sample Preparation

In some aspects, the present application provides advantages for nucleic acid sequencing in biological samples such as cell or tissue samples comprising autofluorescent moieties. In some embodiments, autofluorescence is avoided by not imaging the biological sample using an excitation wavelength to detect fluorescence during one or more cycles of nucleic acid sequencing.

A sample disclosed herein can be or derived from any biological sample. The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, a cell pellet, a cell block, a needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some instances, the biological sample may be provided on a substrate. In some instances, a substrate herein can be any support that is insoluble in aqueous liquid and which allows for positioning of biological samples, analytes, features, and/or reagents (e.g., probes) on the support. In some instances, a biological sample can be attached to a substrate. Attachment of the biological sample can be irreversible or reversible, depending upon the nature of the sample and subsequent steps in the analytical method. In certain instances, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some instances, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some instances, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some instances, the biological sample can be prepared using formalin-fixation and paraffin-embedding (FFPE), which are established methods. In some instances, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some instances, the methods provided herein comprise one or more post-fixing (also referred to as post-fixation) steps. In some instances, one or more post-fixing step is performed after contacting a sample with a polynucleotide disclosed herein, e.g., one or more probes such as a circular or padlock probe. In some instances, one or more post-fixing step is performed after a hybridization complex comprising a probe and a target is formed in a sample. In some instances, one or more post-fixing step is performed prior to a ligation reaction disclosed herein.

In some instances, a method disclosed herein comprises de-crosslinking the reversibly cross-linked biological sample. The de-crosslinking does not need to be complete. In some instances, only a portion of crosslinked molecules in the reversibly cross-linked biological sample are de-crosslinked and allowed to migrate.

In some instances, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some instances, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated by reference herein. Any suitable method for sample permeabilization can generally be used in connection with the samples described herein.

In some instances, the biological sample can be permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some instances, DNase and Rnase inactivating agents or inhibitors such as proteinase K, and/or chelating agents such as EDTA, can be added to the sample. For example, a method disclosed herein comprises a step for increasing accessibility of a nucleic acid for binding, e.g., a denaturation step to open up DNA in a cell for hybridization by a probe. For example, proteinase K treatment may be used to free up DNA with proteins bound thereto.

### Embedding

In some instances, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some instances, the hydrogel is formed such that the hydrogel is internalized within the biological sample. Biological samples can include analytes (e.g., protein, RNA, and/or DNA) embedded in a 3D matrix. In some instances, amplicons (e.g., rolling circle amplification products) derived from or associated with analytes (e.g., protein, RNA, and/or DNA) can be embedded in a 3D matrix. In some instances, a 3D matrix comprises a network of natural molecules and/or synthetic molecules that are chemically and/or enzymatically linked, e.g., by crosslinking. In some instances, a 3D matrix comprises a synthetic polymer. In some instances, a 3D matrix comprises a hydrogel.

In some embodiments, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some instances, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some instances, the hydrogel is formed such that the hydrogel is internalized within the biological sample.

In some instances, the biological sample is immobilized in the hydrogel via cross-linking of the polymer material that forms the hydrogel. Cross-linking can be performed chemically and/or photochemically, or alternatively by any other suitable hydrogel-formation method.

In some instances, the biological sample is reversibly cross-linked prior to or during an in situ assay. In some embodiments, the analytes, polynucleotides and/or amplification product (e.g., amplicon) of an analyte or a probe bound thereto can be anchored to a polymer matrix. For example, the polymer matrix can be a hydrogel. In some instances, one or more of the polynucleotide probe(s) and/or amplification product (e.g., amplicon) thereof can be modified to contain functional groups that can be used as an anchoring site to attach the polynucleotide probes and/or amplification product to a polymer matrix. In some instances, a modified probe comprising oligo dT may be used to bind to mRNA molecules of interest, followed by reversible or irreversible crosslinking of the mRNA molecules.

In some instances, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some instances, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some instances, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, each of which is herein incorporated by reference in its entirety.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, which is herein incorporated by reference in its entirety.

In some instances, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some instances, hydrogel formation occurs after contacting one or more second materials during formation of the substrate. Hydrogel formation can also occur within a structure (e.g., wells, ridges, projections, and/or markings) located on a substrate.

In some instances, hydrogel formation on a substrate occurs before, contemporaneously with, or after probes are provided to the sample. For example, hydrogel formation can be performed on the substrate already containing the probes.

In some instances, hydrogel formation occurs within a biological sample. In some embodiments, a biological sample (e.g., tissue section) is embedded in a hydrogel. In some instances, hydrogel subunits are infused into the biological sample, and polymerization of the hydrogel is initiated by an external or internal stimulus.

In instances in which a hydrogel is formed within a biological sample, functionalization chemistry can be used. In some instances, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some instances, hydrogel formation within a biological sample is permanent. For example, biological macromolecules can permanently adhere to the hydrogel allowing multiple rounds of interrogation. In some instances, hydrogel formation within a biological sample is reversible. In some instances, HTC reagents are added to the hydrogel before, contemporaneously with, and/or after polymerization. In some instances, a cell labeling agent is added to the hydrogel before, contemporaneously with, and/or after polymerization. In some instances, a cell-penetrating agent is added to the hydrogel before, contemporaneously with, and/or after polymerization.

In some instances, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some instances, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some instances, a hydrogel-embedded sample is stored before or after clearing of hydrogel, in a medium (e.g., a mounting medium, methylcellulose, or other semi-solid mediums).

In some instances, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990, each of which is herein incorporated by reference in its entirety. Isometric expansion of the sample can increase the spatial resolution of the subsequent analysis of the sample. The increased resolution in spatial profiling can be determined by comparison of an isometrically expanded sample with a sample that has not been isometrically expanded. In some instances, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some instances, the sample is isometrically expanded to at least 2× and less than 20× of its non-expanded size.

### Staining and Immunohistochemistry (IHC)

To facilitate visualization, biological samples can be stained using a wide variety of stains and staining techniques. In some instances, for example, a sample can be stained using any number of stains and/or immunohistochemical reagents. One or more staining steps may be performed to prepare or process a biological sample for an assay described herein or may be performed during and/or after an assay. In some instances, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some instances, cells in the sample can be segmented using one or more images taken of the stained sample.

In some instances, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some instances, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some instances, the sample can be stained using Romanowsky stain, including Wright's stain, Jenner's stain, Can-Grunwald stain, Leishman stain, and Giemsa stain.

In some instances, biological samples can be destained. Any suitable methods of destaining or discoloring a biological sample may be utilized and generally depend on the nature of the stain(s) applied to the sample. For example, in some instances, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, each of which is herein incorporated by reference in its entirety.

### VI. Analytes

A biological sample may comprise one or a plurality of analytes of interest. Methods for performing multiplexed assays to analyze two or more different analytes in a single biological sample are provided.

The methods and compositions disclosed herein can be used to detect and analyze a wide variety of different analytes. In some embodiments, an analyte can include any biological substance, structure, moiety, or component to be analyzed. In some embodiments, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

### Labeling Agents

In some instances, provided herein are methods and compositions for analyzing endogenous analytes (e.g., RNA, ssDNA, cell surface or intracellular proteins, and/or metabolites) in a sample using one or more labeling agents. In some instances, an analyte labeling agent may include an agent that interacts with an analyte (e.g., an endogenous analyte in a sample). In some instances, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some instances, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some instances, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some instances, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some instances, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of non-limiting examples of labeling agents, reporter oligonucleotides, and methods of use, see, e.g., U.S. Pat. No. 10,550,429; U.S. Pat. Pub. 20190177800; and U.S. Pat. Pub. 20190367969, each of which is herein incorporated by reference in its entirety.

In some instances, an analyte binding moiety includes one or more antibodies or epitope-binding fragments thereof. The antibodies or epitope-binding fragments including the analyte binding moiety can specifically bind to a target analyte. In some instances, the analyte is a protein (e.g., a protein on a surface of the biological sample (e.g., a cell) or an intracellular protein). In some instances, a plurality of analyte labeling agents comprising a plurality of analyte binding moieties bind a plurality of analytes present in a biological sample. In some instances, the plurality of analytes includes a single species of analyte (e.g., a single species of polypeptide). In some instances in which the plurality of analytes includes a single species of analyte, the analyte binding moieties of the plurality of analyte labeling agents are the same. In some instances in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some instances, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some embodiments, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is herein incorporated by reference in its entirety. Likewise, protein and peptide biotinylation techniques have been developed and are readily available. See, e.g., U.S. Pat. No. 6,265,552, which is herein incorporated by reference in its entirety. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some instances, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some instances, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### Generation of Products

In some instances, provided herein are methods and compositions for analyzing one or more products of an endogenous analyte and/or a labeling agent in a biological sample. In some instances, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some instances, a labeling agent that directly or indirectly binds to an analyte in the biological sample is analyzed. In some instances, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### (a) Hybridization

In some instances, a hybridization product comprising the pairing of substantially complementary or complementary nucleic acid sequences within two different molecules can be analyzed. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Non-limiting examples of barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

### (b) Ligation

In some instances, a ligation product of an endogenous analyte and/or a labeling agent can be analyzed. In some instances, the ligation product is formed between two or more endogenous analytes. In some instances, the ligation product is formed between two or more labeling agents. In some instances, the ligation product is an intramolecular ligation of an endogenous analyte. In some instances, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some instances, provided herein is a probe or probe set capable of DNA-templated ligation, such as from a cDNA molecule. See, e.g., U.S. Pat. No. 8,551,710, which is herein incorporated by reference in its entirety. In some instances, provided herein is a probe or probe set capable of RNA-templated ligation. See, e.g., U.S. Pat. Pub. 2020/0224244 which is herein incorporated by reference in its entirety. In some instances, the probe set is a SNAIL probe set. See, e.g., U.S. Pat. Pub. 20190055594, which is herein incorporated by reference in its entirety. In some instances, provided herein is a multiplexed proximity ligation assay. See, e.g., U.S. Pat. Pub. 2014/0194311 which is herein incorporated by reference in its entirety. In some instances, provided herein is a probe or probe set capable of proximity ligation, for instance a proximity ligation assay for RNA (e.g., PLAYR) probe set. See, e.g., U.S. Pat. Pub. 20160108458, which is herein incorporated by reference in its entirety. In some instances, a circular probe can be indirectly hybridized to the target nucleic acid. In some instances, the circular construct is formed from a probe set capable of proximity ligation, for instance a proximity ligation in situ hybridization (PLISH) probe set. See, e.g., U.S. Pat. Pub. 2020/0224243 which is herein incorporated by reference in its entirety.

In some instances, the ligation involves chemical ligation (e.g., click chemistry ligation). In some instances, the chemical ligation involves template dependent ligation. In some instances, the chemical ligation involves template independent ligation. In some instances, the click reaction is a template-independent reaction (see, e.g., Xiong and Seela (2011), J. Org. Chem. 76(14): 5584-5597, which is herein incorporated by reference in its entirety). In some instances, the click reaction is a template-dependent reaction or template-directed reaction. In some instances, the template-dependent reaction is sensitive to base pair mismatches such that reaction rate is significantly higher for matched versus unmatched templates. In some instances, the click reaction is a nucleophilic addition template-dependent reaction. In some instances, the click reaction is a cyclopropane-tetrazine template-dependent reaction.

In some instances, the ligation involves enzymatic ligation. In some instances, the enzymatic ligation involves use of a ligase. In some embodiments, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some instances, the ligase is a T4 RNA ligase. In some instances, the ligase is a splintR ligase. In some instances, the ligase is a single stranded DNA ligase. In some instances, the ligase is a T4 DNA ligase. In some instances, the ligase is a ligase that has an DNA-splinted DNA ligase activity. In some instances, the ligase is a ligase that has an RNA-splinted DNA ligase activity.

In some instances, the ligation herein is a direct ligation. In some instances, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some instances, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific instances, the gap may be a gap of about 1, 2, 3, 4, 5, 6, 7, 8, 9 or 10 or more nucleotides, of any integer (or range of integers) of nucleotides in between the indicated values. In some instances, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some instances, the ligation herein is preceded by gap filling. In other instances, the ligation herein does not require gap filling.

In some instances, ligation of the polynucleotides produces polynucleotides with melting temperature higher than that of unligated polynucleotides. Thus, in some embodiments, ligation stabilizes the hybridization complex containing the ligated polynucleotides prior to subsequent steps, comprising amplification and detection.

In some embodiments, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some instances, the ligation herein is a proximity ligation of ligating two (or more) nucleic acid sequences that are in proximity with each other, e.g., through enzymatic means (e.g., a ligase). In some instances, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, which is herein incorporated by reference in its entirety). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some instances, a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents) can be analyzed.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some instances, a product of an endogenous analyte and/or a labeling agent is an amplification product of one or more polynucleotides, for instance, a circular probe or circularizable probe or probe set. In some instances, the amplifying is achieved by performing rolling circle amplification (RCA). In other instances, a primer that hybridizes to the circular probe or circularized probe is added and used as such for amplification. In some instances, the RCA comprises a linear RCA, a branched RCA, a dendritic RCA, or any combination thereof.

In some instances, the amplification is performed at a temperature between or between about 20° C. and about 60° C. In some instances, the amplification is performed at a temperature between or between about 30° C. and about 40° C. In some embodiments, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some instances, upon addition of a DNA polymerase in the presence of appropriate dNTP precursors and other cofactors, a primer is elongated to produce multiple copies of the circular template. This amplification step can utilize isothermal amplification or non-isothermal amplification. In some instances, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:e1 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801, each of which is herein incorporated by reference in its entirety). Non-limiting examples of polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some embodiments, DNA polymerases that have been engineered or mutated to have desirable characteristics can be employed. In some instances, the polymerase is phi29 DNA polymerase.

In some embodiments, during the amplification step, modified nucleotides can be added to the reaction to incorporate the modified nucleotides in the amplification product (e.g., nanoball). Non-limiting examples of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some embodiments, the amplification products comprises a modified nucleotide, such as an amine-modified nucleotide. In some instances, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some embodiments, the polynucleotides and/or amplification product (e.g., amplicon) can be anchored to a polymer matrix. For example, the polymer matrix can be a hydrogel. In some instances, one or more of the polynucleotide probe(s) can be modified to contain functional groups that can be used as an anchoring site to attach the polynucleotide probes and/or amplification product to a polymer matrix. Non-limiting examples of modification and polymer matrix that can be employed in accordance with the provided instances comprise those described in, for example, WO 2014/163886, U.S. Pat. Nos. 10,494,662, 11,078,520, 11,299,767, 12,264,358, WO 2017/079406, U.S. Pat. No. 10,266,888, U.S. Pat. Pub. 2016/0024555, U.S. Pat. No. 10,494,662, US Pat. Pub. 2018/0251833, U.S. Pat. No. 11,118,220, and U.S. Pat. Pub. No. 2017/0219465, each of which is herein incorporated by reference in its entirety. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some embodiments, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some instances, the provided methods involve embedding the one or more polynucleotide probe sets and/or the amplification products in the presence of hydrogel subunits to form one or more hydrogel-embedded amplification products. In some instances, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some instances, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some instances, the RCA template may comprise the target analyte, or a part thereof, where the target analyte is a nucleic acid, or it may be provided or generated as a proxy, or a marker, for the analyte. In some instances, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some instances, a product herein includes a molecule or a complex generated in a series of reactions, e.g., hybridization, ligation, extension, replication, transcription/reverse transcription, and/or amplification (e.g., rolling circle amplification), in any suitable combination. VI. Fluorescence Detection and Imaging Fluorescence detection

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some instances, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of fluorescent labels and nucleotides and/or polynucleotides conjugated to such fluorescent labels comprise those described elsewhere herein and those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), each of which is herein incorporated by reference in its entirety. In some instances, non-limiting examples of techniques and methods applicable to the provided embodiments comprise those described in, for example, U.S. Pat. Nos. 4,757,141, 5,151,507 and 5,091,519, each of which is herein incorporated by reference in its entirety. In some instances, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), each of which is herein incorporated by reference in its entirety. Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, U.S. Pat. Pub. 2002/0045045 and U.S. Pat. Pub. 2003/0017264, each of which is herein incorporated by reference in its entirety. In some instances, a fluorescent label comprises a signaling moiety that conveys information through the fluorescence absorption and/or emission properties of one or more molecules. Non-limiting examples of fluorescence properties comprise fluorescence intensity, fluorescence lifetime, emission spectrum characteristics and energy transfer.

### Imaging

In some embodiments, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some instances, fluorescence microscopy is used for detection and imaging of the sample. In some embodiments, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be or comprise any microscope that uses fluorescence to generate an image, whether it is a more simple set up like an epifluorescence microscope, or a more complicated design such as a confocal microscope, which uses optical sectioning to achieve better z-axis resolution of the sample to be imaged.

In some instances, confocal microscopy is used for detection and imaging of the sample. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immune-stained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some instances, a method herein comprises subjecting the sample to expansion microscopy methods and techniques. Expansion allows individual targets (e.g., mRNA or RNA transcripts) which are densely packed within a cell, to be resolved spatially in a high-throughput manner. Expansion microscopy techniques are known in the art and can be performed as described in US 2016/0116384 and Chen et al., Science, 347, 543 (2015), each of which is herein incorporated by reference in its entirety. In some instances, the method does not comprise subjecting the sample to expansion microscopy. In some instances, the method does not comprise dissociating a cell from the sample such as a tissue or the cellular microenvironment. In some instances, the method does not comprise lysing the sample or cells therein. In some instances, the method does not comprise embedding the sample or molecules from the sample in an exogenous matrix.

In some cases, analysis is performed on one or more images captured, and may comprise processing the image(s) and/or quantifying signals observed. In some instances, images of signals from different detection channels and/or nucleotide incorporation cycles can be compared and analyzed. In some instances, images of signals (or absence thereof) at a particular location in a sample from different detection channels and/or sequential incorporation cycles can be aligned to analyze an analyte at the location. For instance, a particular location in a sample can be tracked and signal spots from sequential incorporation cycles can be analyzed to detect a target polynucleotide sequence (e.g., a barcode sequence or subsequence thereof) in an analyte at the location. The analysis may comprise processing information of one or more cell types, one or more types of analytes, a number or level of analyte, and/or a number or level of cells detected in a particular region of the sample. In some instances, the analysis comprises detecting a sequence e.g., a barcode sequence present in an amplification product at a location in the sample. In some instances, the number of signals detected in a unit area in the biological sample is quantified. In some instances, the signals detected at a corresponding position in the biological sample in a plurality of images taken at different z positions (e.g., in the depth direction) is quantified and analyzed.

### VII. Barcode Sequences

In some instances, an analyte described herein can be associated with one or more barcode(s), e.g., at least two, three, four, five, six, seven, eight, nine, ten, or more barcodes. Barcodes can be used to spatially-resolve molecular components found in biological samples, for example, within a cell or a tissue sample. A barcode can be attached to an analyte or to another moiety or structure (e.g., a target-specific antibody) in a reversible or irreversible manner. In some embodiments, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, a barcode includes two or more sub-barcodes (or barcode segments) that together function as a single barcode. For example, a polynucleotide barcode can include two or more polynucleotide sequences (e.g., sub-barcodes) that are contiguous or that are separated by one or more non-barcode sequences. In some instances, a barcode may comprise about 1, 2, 3, 4, 5, 6, 7, 8, 9, 10, or more than 10 sub-barcodes (or barcode segments). In some instances, each sub-barcode (or barcode segment) may comprise about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some instances, each non-barcode sequence may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some instances, e.g., in a barcode sequencing method, barcode sequences are detected for identification of other molecules including nucleic acid molecules (DNA or RNA) that are longer than the barcode sequences themselves, as opposed to direct sequencing of the longer nucleic acid molecules. In some instances, an N-mer barcode sequence can comprise up to 4^{N} unique sequences given a sequencing read of N bases, and a much shorter sequencing read may be required for molecular identification compared to non-barcoded sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some instances, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, each of which is herein incorporated by reference in its entirety.

### VIII. Cyclic Array Sequencing in a Flow Cell

In some instances, a method of sequencing as provided herein includes “cyclic array sequencing” of amplified template nucleic acid molecules. Cyclic array flow cell sequencing methods generally involve performing multiple cycles of an enzymatic reaction on an array of spatially-separated oligonucleotide features (e.g., clonally-amplified colonies of template nucleic acid fragments tethered to a support surface, e.g., a flow cell surface). In some instances, the template nucleic acid is modified with known adapter sequence(s) comprising, e.g., amplification and/or sequencing primer binding sites, and then affixed to the support surface (e.g., the lumen surface(s) of a flow cell) in a random or patterned array by hybridization to surface-tethered complementary capture probes (complementary to adapter sequences) on the support surface, clonally amplified, and then probed using the aforementioned sequencing reaction as described herein. In some embodiments, the flow cell sequencing comprises massively parallel sequencing reaction, whereby each enzymatic reaction cycle is used to query only one base (the “interrogation” nucleobase) of the template nucleic acid fragment in each oligonucleotide feature, but thousands to billions of template nucleic acid molecules may be processed in parallel. Performing repeated cycles is then used to progressively identify the nucleic acid sequence of each template nucleic acid molecule based on patterns of detection of a signal or detection of an absence of a signal associated with binding of a nucleotide and polymerase-luciferase fusion protein complex to the template, as detected over the course of multiple reaction cycles.

### Nucleic Acid Extraction

Nucleic acid extraction from cells or other biological samples may be performed using any of a variety of techniques known to those of skill in the art. For example, a typical DNA extraction procedure may comprise: (i) collection of a cell or tissue sample from which DNA is to be extracted, (ii) disruption of cell membranes (i.e., cell lysis) to release DNA and other cytoplasmic components, (iii) treatment of the lysed sample with a concentrated salt solution to precipitate proteins, lipids, and RNA, followed by centrifugation to separate out the precipitated proteins, lipids, and RNA, and (iv) purification of DNA from the supernatant (e.g., using spin columns or paramagnetic beads) to remove detergents, proteins, salts, or other reagents used during the cell membrane lysis step. Non-limiting examples of methods for performing nucleic acid (e.g., DNA and RNA) extraction are described in, for example, Ali et al. (2017) “Current Nucleic Acid Extraction Methods and Their Implications to Point-of-Care Diagnostics”, BioMed Research International 2017:9306564, and Dairawan et al. (2020), “The Evolution of DNA Extraction Methods”, Am J Biomed Sci & Res 8(1):39-45, each of which is herein incorporated by reference in its entirety.

A variety of suitable commercial nucleic acid extraction and purification kits are consistent with the disclosure herein. Examples include, but are not limited to, the QIAamp® kits (for isolation of genomic DNA from human samples) and DNAeasy kits (for isolation of genomic DNA from animal or plant samples) from Qiagen (Germantown, Md.), or the Maxwell® and ReliaPrep™ series of kits from Promega (Madison, Wis.).

### Sequencing Library Preparation

Sequence library preparation may be performed using any of a variety of techniques known to those of skill in the art. Library preparation typically comprises performing the steps of, e.g., end repair, tailing, and ligation of adapter sequences to template nucleic acid fragments. Extracted nucleic acid molecules (e.g., DNA molecule), or fragments thereof, that are typically used as the input for sequencing library preparation often have overhangs containing single-stranded DNA (ssDNA overhangs), breaks in the phosphodiester backbone that exist on just one strand (nicks), and/or ssDNA regions internal to the duplex molecule (ssDNA gaps). End repair reactions (using, e.g., a combination of 3′ exonuclease digestion to remove 3′ overhangs and a strand displacing polymerase reaction using dNTPs to fill nicks and gaps) are used to correct these defects in order to maximize the yield for capturing and sequencing the extracted DNA, and result in the generation of blunt-ended, double-stranded DNA (dsDNA) molecules.

Tailing (e.g., A tailing) is an enzymatic method (using, e.g., a Taq DNA polymerase) for adding a non-templated nucleotide (e.g., an A nucleotide) to the 3′ end of a blunt-ended, double-stranded DNA molecule that facilitates the ligation of the adapter sequences used for sequencing.

One or more adapter sequences may then be ligated to the ends of the end-repaired and tailed template nucleic acid molecules. The adapter sequences may comprise, for example, (i) capture sequences (e.g., the Illumina p5 and p7 adapter sequences) that allow the nucleic acid molecules of the library to bind to a flow cell surface comprising complementary capture probes, (ii) amplification primer binding sites for use in performing reverse transcription and/or for generating clonally-amplified clusters on a flow cell surface, (iii) sequencing primer binding sites (e.g., the Illumina Rd1 and Rd2 sequencing primer binding site sequences) used to initiate sequencing. In addition to amplification and/or sequencing primer binding sites, in some instances the adapters may comprise a barcode sequence, e.g., a sample identification barcode sequence (such as the Illumina Index 1 and Index 2 sample identifier sequences).

Non-limiting examples of methods for performing sequencing library preparation are described in, for example, Head et al. (2014), “Library construction for next-generation sequencing: Overviews and challenges”, BioTechniques 56(1):61-77, and Hess et al. (2020), “Library preparation for next generation sequencing: A review of automation strategies”, Biotechnology Advances 41:107537, the entire contents of each of which are incorporated herein by reference.

### Nucleic Acid Amplification (Including Clonal Amplification)

In some instances, the disclosed methods for performing nucleic acid sequencing (e.g., in vitro and/or flow cell sequencing) may comprise performing one or more steps (e.g., 1, 2, 3, 4, 5, or more than 5) steps of nucleic acid amplification. Amplification reactions with respect to in situ based sequencing methods as described herein are discussed previously. In some instances, for example, one or more steps of nucleic acid amplification may be performed as part of sequencing library preparation and/or following sequencing library preparation. In some instances, one or more steps of nucleic acid amplification (e.g., using a solid-phase amplification technique such as bridge amplification) may be performed after the template molecules of a sequencing library have been tethered to a support surface (e.g., a flow cell surface) to generate clonally-amplified colonies of the tethered template nucleic acid fragments.

In some instances, nucleic acid amplification may be performed to amplify all of the nucleic acid molecules extracted from a biological sample (e.g., using a random primer sequence). In some instances, nucleic acid amplification may be performed to amplify a selected subset of nucleic acid molecules extracted from a biological sample (e.g., using one or more primer sequences designed to hybridize to portions of the sequences for one or more target nucleic acid molecules of interest, or to sequences adjacent thereto).

In some instances, nucleic acid amplification may be performed to amplify an entire sequencing library (e.g., using a primer sequence that hybridizes to a common amplification primer binding site in the sequencing library adapters). In some instances, nucleic acid amplification may be performed to amplify selected portions of the sequencing library (e.g., using one or more primer sequences designed to hybridize to one or more amplification primer binding sites associated with one or more identifier sequences (or barcodes) included in the sequencing library adapters).

Nucleic acid amplification may be performed using any of a variety of nucleic acid amplification techniques known to those of sill in the art, including both thermal and/or isothermal nucleic acid amplification techniques. Examples of suitable thermal nucleic acid amplification techniques include, but are not limited to, polymerase chain reaction (PCR), multiplexed PCR, nested PCR, bridge PCR, reverse transcription PCR (RT-PCR). Examples of suitable isothermal nucleic acid amplification techniques include, but are not limited to, rolling circle amplification (RCA), nucleic acid sequence-based amplification (NASBA), loop-mediated isothermal amplification (LAMP), strand displacement amplification (SDA), helicase-dependent amplification (HDA), nicking enzyme amplification reaction (NEAR), and recombinase polymerase amplification (RPA). Examples of methods for performing nucleic acid amplification are described in, for example, Gill et al. (2008), “Nucleic Acid Isothermal Amplification Technologies—A Review”, Nucleosides, Nucleotides, and Nucleic Acids 27:224-243, Fakruddin et al. (2013), “Nucleic acid amplification: Alternative method of polymerase chain reaction”, J Pharm Bioallied Sci. 5(4): 245-252, and U.S. Pat. No. 8,143,008, the entire contents of each of which are hereby incorporated by reference.

### Nucleotides

In some instances, the disclosed methods for performing nucleic acid sequencing (e.g., in situ and/or flow cell sequencing) may comprise the use of modified versions (e.g., comprising a first functional group or a second function group, as described elsewhere herein) of any of a variety of naturally-occurring nucleotides and/or functional analogs thereof (e.g., nucleotide analogs capable of hybridizing to a nucleic acid sequence in a sequence-specific/correctly base-paired manner) to probe a template nucleic acid sequence. Naturally-occurring nucleotides include deoxyribonucleotides (found in DNA) that comprise a deoxyribose sugar moiety, and ribonucleotides (found in RNA) that comprise a ribose sugar moiety. Naturally-occurring deoxyribonucleotides comprise a nucleobase (or “base”) selected from the group consisting of adenine (A), thymine (T), cytosine (C), or guanine (G). Naturally-occurring ribonucleotides comprise a nucleobase selected from the group consisting of uracil (U), adenine (A), cytosine (C), or guanine (G). In some instances, the nucleotides may be terminated (e.g., reversibly terminated). In some instances, the nucleotides may be conjugated to a detectable label, e.g., a fluorophore. In some instances, the nucleotides may be conjugated to other moieties, e.g., reactive functional groups.

### Primers (or Primer Sequences)

In some instances, the disclosed methods for performing nucleic acid sequencing (e.g., in situ and/or flow cell sequencing) can comprise the use of primer sequences that are complementary to, e.g., a subsequence (or primer binding site) that is part of an endogenous nucleic acid target sequence or a sequence (or primer binding site) that is located at or near a barcode (identifier) sequence associated with a target analyte. In some instances, a primer sequence may be designed to hybridize to a primer binding site associated with a single target analyte sequence and/or an associated target-specific barcode sequence. In some instances, a primer sequence may be designed to hybridize to a sequence (or primer binding site) that is associated with a plurality of target analyte sequences and/or associated target-specific barcode sequences (e.g., at least 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, or more than 1000 target analyte sequences and/or associated target-specific barcode sequences).

### Base-Calling

As noted elsewhere herein, the disclosed methods for performing nucleic acid sequencing (e.g., in situ and/or flow cell sequencing) may comprise inferring the sequence of a template nucleic acid molecule from a series of optical signals (e.g., fluorescence signals) detected in images acquired during a repetitive series of sequencing reaction cycles in a process referred to as “base-calling”. The interplay of sequencing chemistry, opto-fluidics hardware, optical sensors, and signal processing software utilized in sequencing platforms affects the types of errors made during sequencing (see, e.g., Lederberger et al. (2011), “Base-calling for next-generation sequencing platforms”, Brief Bioinform. 12(5): 489-497, which is hereby incorporated by reference in its entirety). The characterization of errors associated with the sequencing process and implementation of chemistry-, imaging-, and/or signal processing software-based methods for minimizing sequence errors are thus important for maximizing the accuracy of sequencing results.

In four-color sequencing-by-synthesis methods, for example, a set of four images—one image for each of four detection channels corresponding to the emission wavelengths for four fluorophores used to label the reversibly terminated nucleotides—are acquired in each sequencing cycle. Processing of the images to detect fluorescence intensity signals produces an intensity quadruple for the location of each sequencing colony on a flow cell surface (or the location of each target analyte, or amplified representation thereof (e.g., an RCP) in the case of in situ sequencing), where each value represents the intensity of the fluorescence signal for the detection channels corresponding to A, C, G and T. Ideally, the channel in which the maximum intensity occurs would be the base that is “called” for a given RCP or sequencing colony (or target analyte) in a given cycle. However, the chemical processes involved in sequencing are imperfect, leading to errors in base-calling (see, e.g., Cacho, et al. (2016), “A Comparison of Base-calling Algorithms for Illumina Sequencing Technology”, Briefings in Bioinformatics 17(5):786-795, which is hereby incorporated by reference in its entirety). In some sequencing-by-synthesis (SBS) platforms, for example, sources of error may include phasing (or lagging; e.g., where the primed template nucleic acid molecules at one or more locations fail to incorporate the next base due to variation in polymerase reaction kinetics), pre-phasing (or leading; e.g., where more than one nucleotide is incorporated in a single cycle due to, e.g., impurities in the reversibly terminated nucleotides), signal decay (due to, e.g., photobleaching and/or loss of template nucleic acid during the sequencing process), and cross-talk (e.g., when two or more fluorophore emission spectra overlap, which may cause a positive correlation between signal intensities measured in the corresponding detection channels).

A variety of statistical approaches have been developed to correct for, or minimize, such errors and generate more accurate base-calls. Examples include, but are not limited to, AYB (Goldman Group, European Molecular Biology Laboratory—European Bioinformatics Institute, Cambridgeshire, UK), and Bustard (Illumina, Inc., San Diego, CA).

### Sequence Reads

The output of the base-calling process applied to optical signals detected in a series of images of a biological sample or flow cell surface acquired during a cycling sequencing process consists of a plurality of sequence reads, e.g., the nucleotide sequences determined for all or a portion of a template nucleic acid molecule (e.g., an endogenous nucleic acid analyte or a barcode sequence associated with a target analyte).

In some instances, the sequence reads generated using the disclosed methods for in situ and/or flow cell sequencing may comprise sequence reads of at least about 10, 20, 30, 40, 50, 60, 70, 80, 90, 100 or more nucleotides or base pairs of the template nucleic acid sequences. In some instances, the sequence reads generated using the disclosed methods may comprise sequence reads of at least about 100, 120, 140, 160, 180, 200, 220, 240, 260, 280, 300, 320, 340, 360, 380, 400, or more than 400 nucleotides or base pairs of the template nucleic acid sequences.

In some instances, the disclosed methods for in situ or flow cell sequencing may generate at least about 100, 200, 300, 400, 500, 600, 700, 800, 900, 1,000, or more sequencing reads per run. In some instances, the disclosed method may generate at least about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 10⁶, 5×10⁶, 10⁷, or more than 10⁷ sequencing reads per run.

### Sequence Assembly & Alignment

In some instances, the disclosed methods for in situ and/or flow cell sequencing may comprise assembly of longer template nucleic acid sequences, e.g., genome fragments or whole genomes, from a plurality of relatively short sequence reads. Sequence assembly may be performed by identifying the overlapping sequences from multiple short sequence reads to assemble longer, contiguous sections of sequence.

In some instances, the disclosed methods for in situ and/or flow cell sequencing may comprise identifying a code word corresponding to a sequence read or an assembled sequence, where the code word is one of a plurality of code words in a codebook that includes assignment of each of the plurality of code words to a target analyte of interest. The sequence read or assembled sequence may thus be used to identify a specific target analyte (based on the corresponding code word) in, e.g., a multiplexed in situ detection or sequencing assay.

In some instances, the disclosed methods for in situ and/or flow cell sequencing may comprise alignment of sequence reads and/or assembled sequences to a known reference sequence or consensus sequence (e.g., the GRCh38 human reference genome (Genome Reference Consortium)) from the same or a similar organism. Alignment to a reference sequence or consensus sequence may be used to identify gaps, errors, or variants in the assembled sequence. Any of a variety of bioinformatics software programs known to those of skill in the art may be used to assemble longer sequences from relatively short sequence reads. Examples include, but are not limited to, DBG2OLC (see, e.g., Ye et al. (2016), “DBG2OLC: Efficient Assembly of Large Genomes Using Long Erroneous Reads of the Third Generation Sequencing Technologies”, Scientific Reports 6:31900, which is hereby incorporated by reference in its entirety), SPAdes (see, e.g., Bankevich et al. (2012), “SPAdes: A New Genome Assembly Algorithm and Its Applications to Single-Cell Sequencing”, J. Computational Biol. 19(5):455-477, which is hereby incorporated by reference in its entirety), SparseAssembler (see, e.g., Ye et al. (2012), “Exploiting Sparseness in de novo Genome Assembly”, BMC Bioinformatics 13(Suppl 6):S1, which is hereby incorporated by reference in its entirety), Fermi (see, e.g., Li (2012), “Exploring Single-Sample SNP and INDEL Calling with Whole-Genome de novo Assembly”, Bioinformatics 28(14):1838-1844, which is hereby incorporated by reference in its entirety), and String Graph Assembler (SGA) (see, e.g., Simpson et al. (2012), “Efficient de novo Assembly of Large Genomes Using Compressed Data Structures”, Genome Res. 22: 549-556, which is hereby incorporated by reference in its entirety).

### IX. Opto-Fluidic Instrument

In some instances, the sequencing methods described herein (e.g., in situ sequence or flow cell sequencing) include using instruments having integrated optics and fluidics modules (“opto-fluidic instruments” or “opto-fluidic systems”) for detecting target molecules (e.g., nucleic acids, proteins, antibodies, etc.) in biological samples (e.g., one or more cells or a tissue sample) as described herein.

In an opto-fluidic instrument, the fluidics module is configured to deliver one or more reagents (e.g., a type of polymerase-luciferase fusion protein, a type nucleotide, buffers, etc.) to the biological sample (e.g., to a sample cartridge within which the biological sample is contained or to a flow cell (e.g., within which nucleic acid molecules extracted from the biological sample have been tethered) and/or to remove spent reagents therefrom. In some instances, one or more sample preparation steps (e.g., fixing, embedding, sample clearing, and/or nucleic acid extraction (in the case that nucleic acid molecules are to be extracted and sequenced in a flow cell)) may be performed prior to the sample being placed on the instrument. In some instances, the fluidics module is configured to deliver one or more further reagents (e.g., primary probe(s) such as circular probe(s) or circularizable probe(s) or probe set(s)) and/or to remove non-specifically hybridized probe(s). In some instances, the fluidics module is configured to deliver one or more detectably labeled probes and optionally intermediate probes to detect the target analytes, or amplified representatives thereof (e.g., RCP(s)) in the biological sample. In some instances, the fluidics module is configured to deliver one or more composition (e.g., a type of polymerase-luciferase fusion protein, a type nucleotide, buffers, etc.) to sequence, e.g., native nucleic acid sequences, barcode sequences associated with target analytes, or amplified copies thereof (e.g., barcode sequences included in RCP(s)) in the biological sample. In some instances, the fluidics module is configured to deliver one or more compositions (e.g., a type of polymerase-luciferase fusion protein, a type nucleotide, buffers, etc.) to a flow cell to sequence, e.g., native nucleic acid sequences, barcode sequences, or amplified copies thereof extracted from the biological sample.

Additionally, the optics module is configured to illuminate the biological sample (or flow cell) with light having one or more spectral emission curves (over a range of wavelengths) and subsequently capture one or more images of emitted light signals from the biological sample (or flow cell) during one or more decoding (e.g., probing or sequencing) cycles. In various instances, the captured images may be processed in real time and/or at a later time to determine the presence of the one or more target molecules in the biological sample, as well as two-dimensional and/or three-dimensional position information associated with each detected target molecule within the biological sample. In various instances, the captured images of a flow cell surface are processed in real time and/or at a later time to determine the sequence of the one or more nucleic acid sequences (e.g., barcode sequences associated with one or more target molecules) that have been extracted from a biological sample. In some embodiment, the optics module further comprises an autofocus mechanism configured to maintain focus at a specified sample plane (e.g., a plane that is perpendicular to the optical axis of an objective lens of the optics module).

Additionally, the opto-fluidics instrument includes a sample module configured to receive (and, optionally, secure) one or more biological samples (e.g., biological samples contained with one or more sample cartridges), or to receive (and, optionally, secure) one or more flow cells. In some instances, the sample module includes an X-Y stage configured to move the biological sample (or flow cell) along an X-Y plane (e.g., perpendicular to the optical axis of an objective lens of the optics module).

In various instances, the opto-fluidic instrument is configured to analyze one or more target molecules (e.g., one or more target RNAs) in their naturally occurring place (i.e., in situ) within the biological sample. In some instances, the opto-fluidic instrument is configured to analyze one or more target RNAs in relative spatial locations within the biological sample. For example, an opto-fluidic instrument may be an in-situ analysis system used to analyze a biological sample and detect target molecules including, but not limited to, DNA, RNA, proteins, antibodies, and/or the like. In some instances, the in situ analysis system is used to detect one or more target RNAs using target-primed rolling circle amplification (RCA) according to the methods disclosed herein.

In various instances, the opto-fluidic instrument may be configured to perform in situ target molecule detection via base-by-base sequencing (e.g., by sequencing an identifier sequence such as a barcode sequence associated with a target molecule) and/or any imaging or target molecule detection technique. That is, for example, an opto-fluidic instrument may include a fluidics module that includes fluids needed for establishing the experimental conditions required for the probing or sequencing of target molecules (or associate barcode sequences) in the sample. Further, such an opto-fluidic instrument may also include a sample module configured to receive the sample, and an optics module including an imaging system for illuminating (e.g., exciting one or more fluorescent probes or dyes such as a DAPI dye within the sample) and/or imaging light signals (e.g., luminescent signals) received from the probed sample. The in-situ analysis system may also include other ancillary modules configured to facilitate the operation of the opto-fluidic instrument, such as, but not limited to, cooling systems, motion calibration systems, etc.

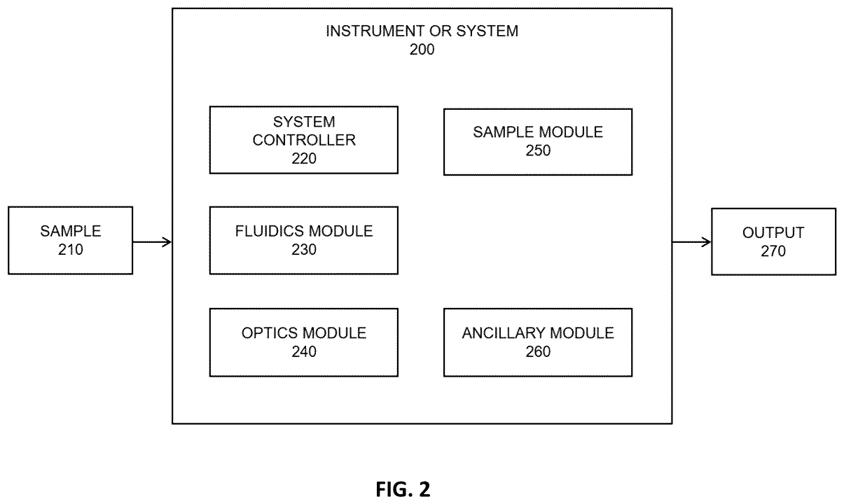

FIG. 2 shows an example workflow of analysis of a biological sample 210 (e.g., cell or tissue sample) using an opto-fluidic instrument or system 200, according to various instances. In various instances, the sample 210 can be a biological sample (e.g., a tissue) that includes molecules such as DNA, RNA, proteins, antibodies, etc. For example, the sample 210 can be a sectioned tissue that is treated to access the RNA thereof for probe (e.g., circularizable probe) hybridization and sequencing (e.g., using a sequencing primer that hybridizes to RCPs to sequence barcode sequences or other marker sequences (e.g., target analyte sequences) in the RCPs) described elsewhere herein.

In various instances, the sample 210 may be placed in the opto-fluidic instrument or system 200 for analysis and detection of the molecules in the sample 210. In various instances, the opto-fluidic instrument or system 200 can be a system configured to facilitate the experimental conditions conducive for the detection of the target molecules. For example, the opto-fluidic instrument or system 200 can include a fluidics module 230, an optics module 240, a sample module 250, and an ancillary module 260, and these modules may be operated by a system controller 220 to create the experimental conditions for hybridization probe-based detection and/or base-by-base sequencing of nucleic acid molecules in the sample 210, as well as to facilitate the imaging of the sample (e.g., by an imaging system of the optics module 240). In various instances, the various modules of the opto-fluidic instrument or system 200 may be separate components in communication with each other, or at least some of them may be integrated together.

In various instances, the sample module 250 may be configured to receive the sample 210 into the opto-fluidic instrument or system 200. For instance, the sample module 260 may include a sample interface module (SIM) that is configured to receive a sample device (e.g., cassette) onto which the sample 210 can be deposited. That is, the sample 210 may be placed in the opto-fluidic instrument or system 200 by depositing the sample 210 (e.g., the sectioned tissue) on a sample device that is then inserted into the SIM of the sample module 250. In some instances, the sample module 250 may also include an X-Y stage onto which the SIM is mounted. The X-Y stage may be configured to move the SIM mounted thereon (e.g., and as such the sample device containing the sample 210 inserted therein) in perpendicular directions along the two-dimensional (2D) plane of the opto-fluidic instrument or system 200.

The experimental conditions that are conducive for the detection of the molecules in the sample 210 may depend on the target molecule detection technique that is employed by the opto-fluidic instrument or system 200. For example, in various instances, the opto-fluidic instrument or system 200 can be a system that is configured to detect molecules (e.g., by detecting hybridization probes that hybridize to nucleic molecules (e.g., barcode sequences) and/or by nucleotides incorporated into extending sequencing primers using an identifier sequence as a template) in the sample 210.

In various instances, the fluidics module 230 may include one or more components that may be used for storing the reagents, as well as for transporting said reagents to and from the sample device containing the sample 210. For example, the fluidics module 230 may include reservoirs configured to store the reagents, as well as a waste container configured for collecting the reagents (e.g., and other waste) after use by the opto-fluidic instrument or system 200 to analyze and detect the molecules of the sample 210. Further, the fluidics module 230 may also include pumps, tubes, pipettes, etc., that are configured to facilitate the transport of the reagent to the sample device (e.g., and as such the sample 210). For instance, the fluidics module 230 may include pumps (“reagent pumps”) that are configured to pump washing/stripping reagents to the sample device for use in washing/stripping the sample 210 (e.g., as well as other washing functions such as washing an objective lens of the imaging system of the optics module 240).

In various instances, the ancillary module 260 can be a cooling system of the opto-fluidic instrument or system 200, and the cooling system may include a network of coolant-carrying tubes that are configured to transport coolants to various modules of the opto-fluidic instrument or system 200 for regulating the temperatures thereof. In such cases, the fluidics module 230 may include coolant reservoirs for storing the coolants and pumps (e.g., “coolant pumps”) for generating a pressure differential, thereby forcing the coolants to flow from the reservoirs to the various modules of the opto-fluidic instrument or system 200 via the coolant-carrying tubes. In some instances, the fluidics module 230 may include returning coolant reservoirs that may be configured to receive and store returning coolants, e.g., heated coolants flowing back into the returning coolant reservoirs after absorbing heat discharged by the various modules of the opto-fluidic instrument or system 200. In such cases, the fluidics module 230 may also include cooling fans that are configured to force air (e.g., cool and/or ambient air) into the returning coolant reservoirs to cool the heated coolants stored therein. In some instance, the fluidics module 230 may also include cooling fans that are configured to force air directly into a component of the opto-fluidic instrument or system 200 so as to cool said component. For example, the fluidics module 230 may include cooling fans that are configured to direct cool or ambient air into the system controller 220 to cool the same.

As discussed above, the opto-fluidic instrument or system 200 may include an optics module 240 which include the various optical components of the opto-fluidic instrument or system 200, such as but not limited to a camera, an illumination module (e.g., LEDs), an objective lens, and/or the like. The optics module 240 may include a fluorescence imaging system that is configured to image the fluorescence emitted by the detectably labeled nucleotides are incorporated in extending sequencing primers in the sample 210 after the detectable labels are excited by light from the illumination module of the optics module 240.

In some instances, the optics module 240 may also include an optical frame onto which the camera, the illumination module, and/or the X-Y stage of the sample module 250 may be mounted.

In various instances, the system controller 220 may be configured to control the operations of the opto-fluidic instrument or system 200 (e.g., and the operations of one or more modules thereof). In some instances, the system controller 220 may take various forms, including a processor, a single computer (or computer system), or multiple computers in communication with each other. In various instances, the system controller 220 may be communicatively coupled with data storage, set of input devices, display system, or a combination thereof. In some cases, some or all of these components may be considered to be part of or otherwise integrated with the system controller 220, may be separate components in communication with each other, or may be integrated together. In other examples, the system controller 220 can be, or may be in communication with, a cloud computing platform.

In various instances, the opto-fluidic instrument or system 200 may analyze the sample 210 and may generate the output 270 that includes indications of the presence of the target molecules in the sample 210. For instance, with respect to instances discussed above where the opto-fluidic instrument or system 200 employs a sequencing technique for detecting molecules, the opto-fluidic instrument or system 200 may cause the sample 210 to undergo successive sequencing cycles, where during the same sequencing cycle the sample is imaged to detect signals associated with nucleotide binding and/or incorporation events at some locations in the sample 210, as well as to detect an absence of signals at other locations in the sample. In such cases, the output 270 may include a series of optical signals (e.g., a code word) specific to each identifier sequence (e.g., a barcode sequence), which allow the identification of the target molecules.

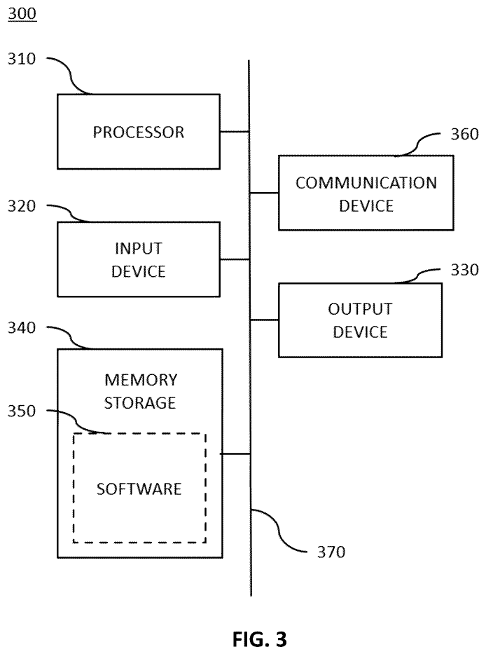

FIG. 3 illustrates an example of a computing device or system in accordance with one or more examples of the disclosure. Device 300 can be a host computer connected to a network. Device 300 can be a client computer or a server. As shown in FIG. 3, device 300 can be any suitable type of microprocessor-based device, such as a personal computer, workstation, server, or handheld computing device (portable electronic device), such as a phone or tablet. The device can include, for example, one or more of processor 310, input device 320, output device 330, memory/storage 340, and communication device 360. Input device 320 and output device 330 can generally correspond to those described above, and they can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage, such as an electrical, magnetic, or optical memory including a RAM, cache, hard drive, or removable storage disk. Communication device 360 can include any suitable device capable of transmitting and receiving signals over a network, such as a network interface chip or device. The components of the computer can be connected in any suitable manner, such as via a physical bus 370 or wirelessly.

Software 350, which can be stored in memory/storage 340 and executed by processor 310, can include, for example, the programming that embodies the functionality of the present disclosure (e.g., as embodied in the methods and systems described above). Software 350 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 340, that can contain or store programming for use by or in connection with an instruction execution system, apparatus, or device.

Software 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 300 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can implement any operating system suitable for operating on the network. Software 350 can be written in any suitable programming language, such as C, C++, Java, or Python. In various implementations, application software embodying the functionality of the present disclosure can be deployed in different configurations, such as in a client/server arrangement or through a web browser as a web-based application or web service, for example.

### X. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of (a), (b), etc., or (i), (ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Generating a Stalled Ternary Complex Comprising a Polymerase-Luciferase Fusion and Nucleotide to Identify a Complementary Nucleotide in a Template Nucleic Acid

This example provides a workflow for identifying a base in a template nucleic acid using a polymerase-luciferase fusion protein and an unlabeled nucleotide.

A synthetic gene is embedded in a hydrogel matrix on a glass slide. The hydrogel is then contacted with a circularizable probe that hybridizes to the pseudogene and includes a barcode sequence. The circularizable probe is allowed to hybridize to the synthetic gene. The hydrogel is then contacted with a ligation reaction mix including ligase, and the circularizable probe is ligated to form a circular template for rolling circle amplification (RCA). The hydrogel is then incubated with an RCA mixture containing a DNA polymerase and dNTP for RCA of the circularized probes. From this amplification, the RCA product (e.g., RCP) comprises the template nucleic acid molecule comprising multiple copies of a barcode sequence adjacent to a sequencing primer-binding sequence. A sequencing primer is hybridized to the sequencing primer-binding sequence to allow sequencing of the copies of the barcode sequence in the RCP.

Next, a dye-linked primer is hybridized to the RCP to identify the location of the RCP on the slide. A composition comprising a polymerase-luciferase fusion protein and a single type of unlabeled nucleotide is contacted with the biological sample in a buffer including calcium. The buffer does not comprise a catalytic metal ion cofactor of the polymerase (e.g, magnesium).

When the composition is contacted with the sample in the presence of a single type of nucleotide that is complementary to the first base of the barcode sequence, a stable ternary complex forms between the polymerase-luciferase fusion protein, the complementary nucleotide, and the template nucleic acid. The absence of a catalytic metal ion cofactor of the polymerase prevents incorporation of the complementary nucleotide. In the presence of the luciferase's substrate, luminescence is detected at the identified location of the RCP. Advantageously, detection of the luminescence does not require fluorescent excitation, and avoids problems associated with fluorescent imaging such as singlet oxygen generation, photobleaching, and tissue autofluorescence.

### Example 2: Sequencing Using Different Types of Polymerase-Luciferase Fusion Proteins In Situ

This example provides a workflow for sequentially flowing in a first type of free nucleotide and a first type of polymerase-luciferase fusion, flowing in a second type of free nucleotide and a second type of polymerase-luciferase fusion, flowing in a third type of free nucleotide and a third type of polymerase-luciferase fusion, flowing in a fourth type of free nucleotide and a fourth type of polymerase-luciferase fusion, while maintaining conditions that stabilize a ternary complex, then detecting luminescence from the bound polymerase-luciferase fusions, where each of the four different types of polymerase-luciferase fusion has a luciferase that emits a visually distinct luminescent signal indicating the presence of the nucleotide type that was flowed in the same step as the corresponding polymerase-luciferase fusion, therefore detecting one or more nucleotides. An example workflow is shown in FIG. 1. An example of a polymerase-luciferase fusion protein, comprising a luciferase domain from Lingulodinium polyedrum (a blue luciferase) and a polymerase domain from bacterial DNA polymerase II. Use of polymerase-luciferase fusion proteins for generating nucleotide-specific signal readouts may provide certain advantages such as allowing for the use of standard dNTPs for sequencing-by-synthesis instead of complex reversible terminators. Because luminescence is produced by luciferases bound to a substrate, no excitation light or filters are required, and background autofluorescence is not a concern. Additionally, the sequencing process can be performed under cool or room temperature conditions without requiring heat to strip between cycles.

A tissue sample is obtained and cryosectioned onto a glass slide for processing. The tissue is fixed by incubating in 3.7% paraformaldehyde (PFA). To prepare for probe hybridization, a wash buffer is added to the tissue sample. The washed tissue sample is then contacted with a circularizable probe comprising a template nucleic acid molecule and a barcode sequence. The barcode sequence identifies a target analyte within the tissue sample. The circularizable probe is allowed to hybridize to the target analyte. The tissue sample is then contacted with a ligation reaction mix including ligase, and the circularizable probe is ligated to form a circular template for rolling circle amplification (RCA). The tissue sample is then incubated with an RCA mixture containing a Phi29 DNA polymerase and dNTP for RCA of the circularized probes. From this amplification, the RCA product (e.g., RCP) comprises the template nucleic acid molecule comprising multiple copies of a barcode sequence adjacent to a sequencing primer-binding site.

A non-limiting example of the disclosed sequencing methods is illustrated schematically in FIG. 1. In the case of in situ sequencing, the tissue sample is washed and then contacted with a sequencing primer. The sequencing primer is allowed to hybridize to the template nucleic acid molecule in the RCP at the sequencing primer binding site adjacent to the barcode sequence. The tissue sample is washed and then contacted (in one or more steps) with a first type of polymerase-luciferase fusion protein (e.g., a polymerase-luciferase fusion comprising a luciferase with a green bioluminescent signal) and a first type of deoxynucleotide triphosphate (dNTP) (selected from dATP, dTTP, dGTP, and dCTP) complementary to a first type of nucleotide in the template strand (selected from A, T, G, and C), in an incorporation buffer comprising calcium ions (step 1 in FIG. 1). The presence of calcium instead of magnesium in the buffer promotes stabilization of ternary complexes. A dNTP of the first type of dNTPs and a polymerase-luciferase fusion protein of the first type of polymerase-luciferase fusion proteins are allowed to form a stalled ternary complex with a nucleotide of the first nucleotide type within the template nucleic acid molecule. Under suitable conditions (e.g., in an incorporation buffer comprising calcium) a stable ternary complex is formed between the nucleotide within the template nucleic acid molecule, the dNTP and the polymerase domain of the polymerase-luciferase fusion product. The tissue sample is then washed to remove unbound fusion proteins of the first polymerase-luciferase fusion proteins and unbound dNTPs of the first set of dNTPs, while stable ternary complexes are maintained.

The tissue sample is then contacted with a second type of polymerase-luciferase fusion protein (e.g., a polymerase-luciferase fusion comprising a luciferase domain with a red bioluminescent signal) and a second type of dNTP (selected from dATP, dTTP, dGTP, and dCTP, and different from the first type of dNTP) complementary to a second type of nucleotide in the template strand (selected from A, T, G, and C, and different from the first type of nucleotide) (step 2 in FIG. 1). The contacting again occurs in an incorporation buffer that comprises calcium ions and promotes stabilization of ternary complexes. A dNTP of the second type of dNTPs and a polymerase-luciferase fusion protein of the second type of polymerase-luciferase fusion are allowed to form a stalled ternary complex with a nucleotide of the second nucleotide type within the template nucleic acid molecule. The tissue sample is then washed to remove unbound polymerase-luciferase fusion proteins and nucleotide molecules of the second set of dNTPs (e.g., polymerase-luciferase fusion proteins and dNTPs that are not in stable ternary complexes are washed away, while polymerase-luciferase fusion proteins and dNTPs of the first and the second types that are in stable ternary complexes remain contacted with the tissue sample).

The tissue sample is then contacted (in one or more steps) with a third type of polymerase-luciferase fusion protein (e.g., a polymerase-luciferase fusion protein comprising a luciferase domain with a blue bioluminescent signal, such as the example polymerase-luciferase fusion protein in FIG. 2 comprising a luciferase domain from L. polyedrum and a polymerase domain from bacterial DNA polymerase II) and a third type of dNTP (selected from dATP, dTTP, dGTP, and dCTP, and different from the first and second types of dNTP) complementary to a third type of nucleotide in the template strand (selected from A, T, G, and C, and different from the first and second types of nucleotide) (step 3 of FIG. 1). The contacting occurs in an incorporation buffer comprising calcium ions. A dNTP of the third set of dNTPs and a polymerase-luciferase fusion protein of the third type of polymerase-luciferase fusion protein are allowed to form a stalled ternary complex with a nucleotide of the third nucleotide type within the template nucleic acid molecules. The tissue sample is then washed to remove unbound nucleotide molecules of the third set of dNTPs, while stable ternary complexes are maintained.

The tissue sample is then contacted (in one or more steps) with a fourth type of polymerase-luciferase fusion protein (e.g., a polymerase-luciferase fusion protein comprising a luciferase domain with a far-red bioluminescent signal such as a luciferase derived from P. hirtus, for example, as described in Viviani et al, Int. J. Mol. Sci. 2021, 22, 303, which is herein incorporated by reference in its entirety) and a fourth type of dNTP (selected from dATP, dTTP, dGTP, and dCTP, and different from the first, second, and third types of dNTP) complementary to a fourth type of nucleotide in the template strand (selected from A, T, G, and C, and different from the first, second, and third types of nucleotide) (step 4 in FIG. 1). The contacting occurs in an incorporation buffer comprising calcium ions. A dNTP of the fourth set of dNTPs and a polymerase-luciferase fusion protein of the fourth type of polymerase-luciferase fusion proteins are allowed to form a stalled ternary complex with a nucleotide of the fourth nucleotide type within the template nucleic acid molecules. The tissue sample is then washed to remove unbound nucleotide molecules of the fourth set of dNTPs.

A mixture comprising a substrate for each of the first, second, third, and fourth luciferases of the four polymerase-luciferase fusion proteins is then washed onto the sample, while conditions promoting stabilization of ternary complexes are maintained. The presence of the substrate causes each polymerase-luciferase fusion protein to behave as its own photocell and fluoresce in the color corresponding to the matching dNTP.

The tissue sample is then imaged to detect a bioluminescent photosignal emitted by the activated (i.e., substrate-bound) first, second, third, and fourth polymerase-luciferase fusion proteins forming stalled ternary complexes with first, second, third, and fourth types of dNTPs complementary to first, second, third, and fourth types of nucleotides within the template nucleic acid molecules (step 5 in FIG. 1). Imaging is used to detect a bioluminescent signal associated with the first type of polymerase-luciferase fusion protein (e.g., a polymerase-luciferase fusion with a yellow-green bioluminescent signal) nucleotide molecule corresponding to the first type of dNTP, a bioluminescent signal associated with the second type of polymerase-luciferase fusion protein (e.g., a polymerase-luciferase with a red bioluminescent signal) corresponding to the second type of dNTP, a bioluminescent signal associated with the third type of polymerase-luciferase fusion protein (e.g., a polymerase-luciferase fusion protein with a blue bioluminescent signal) corresponding to the third type of dNTP, and/or a bioluminescent signal associated with the fourth type of polymerase-luciferase fusion protein (e.g., a polymerase-luciferase fusion protein with a far-red bioluminescent signal) corresponding to the fourth type of dNTP. Fluorescence microscopy is not required to detect the luciferase signals, as the luciferase domains emit visible bioluminescent signals at distinct wavelengths when bound to a substrate in the absence of an excitation signal.

The tissue sample is bathed in a magnesium buffer, thus displacing the calcium ions in the binding pocket of the polymerase of the polymerase-luciferase fusion proteins and incorporating the trapped dNTPs in the ternary complexes into the growing primer strand. All small molecule substrates are washed away with the polymerase-luciferase fusion proteins once the ternary complexes are liberated, and the cycle is then repeated (step 6 in FIG. 1).

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

What is claimed is:

1. A method for sequencing a template nucleic acid molecule comprising:
(a-i) contacting a sample comprising a priming strand bound to the template nucleic acid molecule with a first type of polymerase-luciferase fusion protein and a first type of nucleotide under conditions that stabilize a ternary complex between the first type of polymerase-luciferase fusion protein, the template nucleic acid molecule, and the first type of nucleotide when the first type of nucleotide is complementary to the template nucleic acid at a base adjacent to the bound priming strand;
(a-ii) contacting the sample with a second type of polymerase-luciferase fusion protein and a second type of nucleotide under conditions that stabilize a ternary complex between the second type of polymerase-luciferase fusion protein, the template nucleic acid molecule, and the second type of nucleotide when the second type of nucleotide is complementary to the template nucleic acid at the base adjacent to the bound priming strand; and
(b) imaging the sample to detect luminescence, wherein:
the first type of polymerase-luciferase fusion comprises a first type of luciferase that emits a first type of luminescent signal indicating formation of a ternary complex comprising the first type of nucleotide, and
the second type of polymerase-luciferase fusion comprises a second type of luciferase that emits a second type of luminescent signal indicating formation of a ternary complex comprising the second type of nucleotide;
thereby identifying the base of the template nucleic acid adjacent to the bound priming strand from the detected luminescence.

2. The method of claim 1, wherein the method further comprises:
(a-iii) contacting the sample with a third type of polymerase-luciferase fusion protein and a third type of nucleotide under conditions that stabilize a ternary complex between the third type of polymerase-luciferase fusion protein, the template nucleic acid molecule, and the third type of nucleotide when the third type of nucleotide is complementary to the template nucleic acid at the base adjacent to the bound priming strand
wherein the third type of polymerase-luciferase fusion comprises a third type of luciferase that emits a third type of luminescent signal indicating formation of a ternary complex comprising the third type of nucleotide.

3. The method of claim 2, wherein the method further comprises:
(a-iv) contacting the sample with a fourth type of nucleotide and a polymerase under conditions that stabilize a ternary complex between the polymerase, the template nucleic acid molecule, and the fourth type of nucleotide when the fourth type of nucleotide is complementary to the template nucleic acid at the base adjacent to the bound priming strand.

4. The method of claim 2, wherein the method further comprises:
(a-iv) contacting the sample with a fourth type of polymerase-luciferase fusion protein and a fourth type of nucleotide under conditions that stabilize a ternary complex between the fourth type of polymerase-luciferase fusion protein, the template nucleic acid molecule, and the fourth type of nucleotide when the fourth type of nucleotide is complementary to the template nucleic acid at the base adjacent to the bound priming strand;
wherein the fourth type of polymerase-luciferase fusion comprises a fourth type of luciferase that emits a fourth type of luminescent signal indicating formation of a ternary complex comprising the fourth type of nucleotide.

5. The method of claim 1, wherein the first and second types of luciferase are different types of luciferase independently selected from the group consisting of a yellow-emitting luciferase, a green-emitting luciferase, a blue-emitting luciferase, a red-emitting luciferase, and a far-red-emitting luciferase.

6-7. (canceled)

8. The method of claim 1, wherein the ternary complex-stabilizing conditions are maintained throughout steps (a-i), (a-ii), and step (b).

9-10. (canceled)

11. The method of claim 1, wherein the ternary complex-stabilizing conditions comprise presence of a non-catalytic metal ion.

12. The method of claim 1, wherein the ternary complex-stabilizing conditions comprise absence of a catalytic metal ion cofactor of the polymerase.

13. The method of claim 12, wherein the catalytic metal ion is magnesium or manganese.

14. The method of claim 11, wherein the non-catalytic metal ion is calcium, strontium, tin, or nickel.

15. The method of claim 1, wherein the method comprises washing the sample to remove unbound polymerase-luciferase fusion proteins and unbound nucleotides prior to performing the detecting step.

16. The method of claim 15, wherein the washing is performed with a wash buffer comprising a non-catalytic metal ion.

17. The method of claim 1, wherein the method comprises washing the sample between each of the contacting steps to remove unbound polymerase-luciferase fusion proteins and unbound nucleotides.

18. The method of claim 17, wherein the washing is performed with a wash buffer comprising a non-catalytic metal ion.

19. The method of claim 15, wherein the washing is performed with a wash buffer that does not comprise a catalytic metal cation of the polymerase.

20. The method of claim 1, wherein after imaging the sample to detect luminescence, the method comprises extending the priming strand by incorporating a reversibly terminated nucleotide, and repeating steps (a-i), (a-ii), and (b) for at least one additional cycle to identify at least one additional base of the template nucleic acid strand.

21-23. (canceled)

24. The method of claim 3, wherein the first type of nucleotide, second type of nucleotide, third type of nucleotide, and fourth type of nucleotide comprise different nucleobases selected from the group consisting of A, T, C, and G.

25. The method of claim 1, wherein the first type of nucleotide and second type of nucleotide are not reversibly terminated nucleotides.

26-30. (canceled)

31. The method of claim 1, wherein the template nucleic acid molecule comprises a target analyte nucleic acid molecule.

32. The method of claim 1, wherein the template nucleic acid molecule comprises a sequence of interest associated with a target analyte.

33. The method of claim 32, further comprising hybridizing a circularizable probe to the target analyte or to a labeling agent bound to the target analyte and ligating the circularizable probe to form a circularized probe, wherein the method further comprises performing rolling circle amplification of the circularized probe to generate the template nucleic acid molecule.

34. (canceled)

35. The method of claim 33, wherein the target analyte nucleic acid molecule comprises an mRNA molecule.

36. The method of claim 1, wherein the template nucleic acid molecule to be sequenced is attached to a solid support.

37. The method of claim 36, wherein the solid support comprises a sequencing flow cell.

38. The method of claim 1, wherein the template nucleic acid molecule is sequenced in situ in a cell sample or tissue sample.

39. The method of claim 38, wherein the cell sample comprises a layer of cells deposited on a surface.

40-44. (canceled)
